(19) United States
(12) Patent Application Publication
(10) Pub. No.: US 2025/0059471 A1
(43) Pub. Date: Feb. 20, 2025

(54) WATER-SOLUBLE GRAFT POLYMER, THEIR PREPARATION, USES, AND COMPOSITIONS COMPRISING SUCH POLYMERS

(71) Applicant: BASF SE, Ludwigshafen am Rhein (DE)

(72) Inventors: Stephan BAUER, Ludwigshafen am Rhein (DE); Johannes MAUL, Ludwigshafen am Rhein (DE); Thomas Alexander KROENER, Ludwigshafen am Rhein (DE); Claudia ESPER, Ludwigshafen am Rhein (DE); Keith E. GUTOWSKI, Wyandotte, MI (US); Daniel Scott NIEDZWIECKI, Florham Park, NJ (US); Alexandros LAMPROU, Shanghai (CN)

(21) Appl. No.: 18/721,211

(22) PCT Filed: Dec. 14, 2022
(86) PCT No.: PCT/EP2022/085740

§ 371 Date: Jun. 18, 2024

(30) Foreign Application Priority Data
Dec. 22, 2021 (EP) 21216989.0
Jan. 31, 2022 (EP) 22154254.1

(51) Int. Cl. C11D 3/37; C08F 251/00

(52) U.S. Cl. CPC C11D 3/3788; C08F 251/00

(57) ABSTRACT

This invention deals with water-soluble graft polymers comprising as polymer base a polysaccharide, and as grafted side chains at least one water soluble ethylenically unsaturated monomer, such unsaturated monomer comprising at least one sodium carboxylate unit, being produced by radically initiated polymerization reaction, and further employing during the polymerization at least one organic compound capable of complexing metal ions, preferably iron, containing in its structure at least one carboxyl-group which may be partially or fully present as acid-group or deprotonated in salt-form, their manufacture and uses, for example in laundry or dishwashing, and a method of stabilizing such graft polymers. The graft polymers have a solid content after manufacturing of at least 20% and with at least 70% of the solid content. The aqueous solution of a grafted polymer is the characterized by a Gardner color less than 4.0 and a color stability at least 6 months with an increase of maximum one Gardner color value unit at room temperature.

This invention deals with water-soluble graft polymer comprising as polymer base a polysaccharide, and as grafted side chains at least one water soluble ethylenically unsaturated monomer, such unsaturated monomer comprising at least one sodium carboxylate unit, being produced by radically initiated polymerization reaction, and further employing during the polymerization at least one organic compound capable of complexing metal ions, preferably iron, containing in its structure at least one carboxyl-group which may be partially or fully present as acid-group or deprotonated in salt-form. (in this present invention abbreviated as “graft polymer” or “inventive polymer” or “polymer of the invention” whenever the inventive polymers are meant), their manufacture, their uses, particularly for use in cleaning compositions such as laundry and dish washing detergent compositions. The graft polymers have a solid content after manufacturing of at least 20% and with at least 70% of the solid content. The aqueous solution of a grafted polymer is the characterized by a Gardner color less than 4.0 and a color stability at least 6 months with an increase of maximum one Gardner color value unit at room temperature.

Detergent formulators are continuously faced with the task of developing improved products to remove a broad spectrum of soils and stains from fabrics and hard surfaces. Chemically and physico-chemically, the varieties of soils and stains range the spectrum from polar soils, such as proteinaceous, clay, and inorganic soils, to non-polar soils, such as soot, carbon-black, by-products of incomplete hydrocarbon combustion, and organic soils like sebum. These challenges have been accentuated by the recent high interest and motivation to reduce the level of surfactants in cleaning detergents for environmental, sustainability and cost reasons. The reduction of level of surfactants, especially oil-derived surfactants, such as linear alkyl benzene sulfonate, LAS, has typically been found to lead to an erosion of oily/fatty stain removal.

Additionally, the global trend of using washing conditions at lower temperature further diminishes grease cleaning capabilities of typical detergents, since the class of oily and fatty stains shows the greatest performance drop when the temperature is decreased.

Another global trend is the compaction of laundry and dish wash detergents, in order to improve the sustainability in terms of water usage and/or transportation costs, as well as to improve the convenience for the end consumer (e.g., single mono dose products, tabs, pouches and the like), which leads to a high market demand for new raw materials that have a higher weight-efficiency and a significantly broader performance profile.

A further strongly emerging trend is the desire to improve the “footprint” of any product, be it in terms of its origin like being from natural or renewable resources, or all compared to previous products—its production in terms of production efficiency and thus reduced usage of energy, its efficiency in usage such as reduced amounts for the same performance or higher performance at the same amount levels used, its persistence in the natural environment upon and/or after its usage such as bio-degradation.

Hence, due to the climate change, one of the most important targets of for example the detergent and cleaner (D&C) industry today is to significantly lower the CO2 emission per wash, by improving cold water conditions, improving the cleaning efficiency at low temperatures of below 40, 30 or 20 or even colder, and to lower the amounts of chemicals employed per wash, and increasing the weight-efficiency of the cleaning technologies. Another technical hurdle is the reduction of glass corrosion in automatic dish washing (ADW) to generally extend the lifetime of dishware and thereby to contribute to an overall improved sustainability. Another important target FIGURES:4 of the D&C industry is the need for biodegradable polymers, to improve the sustainability of the detergent formulations and to avoid the accumulation of non-degradable polymers or polymer blocks resulting from incomplete biodegradation of polymers (persistent blocks) in the ecosystem, thus lowering the persistence in nature after usage of the materials.

As a result of these trends, there is a strong need for new biodegradable cleaning polymers that provide both excellent primary (i.e., soil removal) and secondary (i.e., whiteness maintenance) cleaning benefits for both hydrophobic and hydrophilic stains, and that do not contain any persistent block within their macromolecular architecture. The materials should exhibit good soil removal for oily/fatty and particulate stains and should also lead to improved whiteness maintenance, minimizing the amount of suspended and emulsified oily/fatty and particulate soil from redepositing on the surfaces of the textiles or hard surfaces. Preferably, the new ingredients would also display a synergy with other cleaning polymers known for improving solely the oily/fatty or particulate stain removal and/or whiteness of fabrics and hard surfaces, leading to further improved detergent compositions.

As a result of these trends the need is evolving for new biodegradable polymers that do not contain any persistent block within their macromolecular architecture.

Graft polymers on polysaccharides using acrylic acid are known as such. They pose however the problem that the colour and the odor of those polymers is rarely acceptable, as they usually are at least yellow to brown with a very typical, often strong odor.

Hence, there was a need to improve such polymers by providing colorless or almost colorless polymer and polymer solutions, exhibiting only faint odor.

The object of the present invention is to provide novel water-soluble graft polymer comprising as polymer base a polysaccharide, and as grafted side chains at least one water soluble ethylenically unsaturated monomer, such unsaturated monomer comprising at least one sodium carboxylate unit, being produced by radically initiated polymerization reaction, and further employing during the polymerization at least one organic compound capable of complexing metal ions, preferably iron, containing in its structure at least one carboxyl-group which may be partially or fully present as acid-group or deprotonated in salt-form.

The following Embodiments 1 to 43 define the invention together with the experiments detailed in the experimental section and the claims:

### Embodiment 1

Water-soluble graft polymer comprising as polymer base a polysaccharide, and as grafted side chains at least one water soluble ethylenically unsaturated monomer, such unsaturated monomer comprising at least one sodium carboxylate unit, being produced by radically initiated polymerization reaction, and further employing during the polymerization at least one organic compound capable of complexing metal ions, preferably iron, containing in its structure at least one carboxyl-group which may be partially or fully present as acid-group or deprotonated in salt-form.

### Embodiment 2

Graft polymer according to Embodiment 1 wherein the polysaccharide consists of a maximum of n=30 glucose units in average.

### Embodiment 3

Graft polymer according to Embodiment 2 wherein the polysaccharide consists of a maximum of n=2-20 glucose units in average.

### Embodiment 4

Graft polymer according to Embodiment 3 wherein the polysaccharide consists of a maximum of n=3-10 glucose units in average.

### Embodiment 5

Graft polymer according to Embodiment 1-4 wherein the polysaccharide is selected from maltodextrin, corn syrups and/or corn syrups solids.

### Embodiment 6

Graft polymer according to any of Embodiments 1-5 wherein the ethylenically unsaturated monomer comprises at least 50 mol. %, preferably at least 90 mol. % sodium acrylate and/or methacrylate, more preferably sodium acrylate, based on the total amount of monomers employed, and optionally other monomers polymerizable with the before mentioned monomers.

### Embodiment 7

Graft polymer according to any of Embodiments 1-6, wherein the radical polymerization is of the radical polymerization-type, preferably radical redox polymerization-type.

### Embodiment 8

Graft polymer according to any of Embodiments 1-7, wherein at least one organic compound is employed to reduce or—preferably—prevent the discoloration before, during and/or after polymerization, more preferably before and/or during polymerization, even more preferably before and during polymerization, and most preferably such that a sufficient amount of the at least one such organic compound is still present after the polymerization and after post-polymerization; “sufficient amount” being defined as “the molar ratio of the total amount of the metal salt and the total amount of the at least one organic compound being between 1:1 to 1:10,000, preferred 1:2 to 1:1,000, mostly preferred between 1:5 to 1:500, such as 1:10 to 1:400, 1:30 to 1:200, 1:50 to 1:200, or about 1:80 to 1:120”.

### Embodiment 9

Graft polymer according to any of Embodiments 1 to 8, wherein as radical initiators for at least the main polymerization stage, a polymerization system comprising at least one metal salt and peroxide, preferably hydrogen peroxide, is employed.

### Embodiment 10

Graft polymer to according to any of Embodiments 9, wherein the at least one organic compound comprises at least one functional group to form metal complexes with the at least one metal salt used as part of the polymerization-system.

### Embodiment 11

Graft polymer according to any of Embodiments 9-10, wherein the metal salt comprises, preferably contains only metal cations, preferably being selected from Fenton catalyzing metals, even more preferably iron and/or copper, which is most preferably employed as ferrous (II) sulfate, copper (II) sulfate, and their hydrate derivatives, even more preferably ferrous (II) sulfate, most preferably employed as ferrous sulfate heptahydrate.

### Embodiment 12

Graft polymer according to any of Embodiments 1-11, wherein the at least one water soluble organic compound—which is preferably of natural origin and more preferably from a renewable resource—contains at least one carboxylate group, such as gluconic acid and their salts, preferably at least two carboxylate groups (COOR), in case of at least two carboxylate groups being more preferably separated with 1 to 3 others atoms, and even more preferably are separated with 1 to 3 others atoms which are selected from the atom types C, Si, N, P, O, S, and even more preferred have one of the following chemical Structures 1 to 4

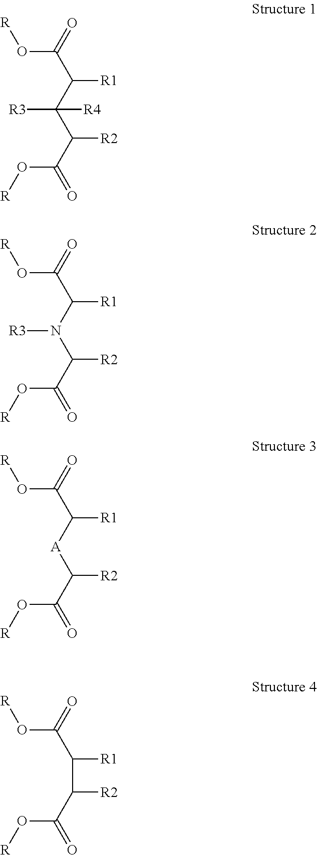

for all Structures 1 to 4 the variable being selected as follows:
R H, Li, Na, K, Ca, preferred H or Na
A O or S
R1, R2, R3: H and/or organic substituent containing 1 to 20 carbon-atoms and optionally further containing amino, hydroxy, carboxylic acid, sulfonic-acid, sulfonate, phosphonate, keto, aldehyde, and/or aromatic groups, preferably H and/or the organic structure containing one or more further carboxylate groups selected from —COOR and —COOH, preferably —COOR
R4 H, OH, or organic substituent containing 1 to 20 carbon-atoms and optionally further containing amino, hydroxy, carboxylic acid, sulfonic-acid, sulfonate, phosphonate, keto, aldehyde, and/or aromatic groups, preferably H and/or the organic structure containing one or more further carboxylate groups selected from —COOR and —COOH, preferably —COOR.

### Embodiment 13

Graft polymer according to Embodiment 12 wherein the organic compound has at least three carboxylate groups (COOR).

### Embodiment 14

Graft polymer according to any of Embodiments 12 to 13, wherein the organic compound is selected from the group consisting of citric acid, metylglycidine di acetic acid, ethylene diamin tetra acetic acid, tetra N,N-bis(carboxylatomethyl)-glutaminic acid, di ethylene triamine penta acetic acid, tartaric acid, succinic acid, or any of their salts such as ammonium, alkali, and/or earth alkali salts such as sodium, potassium, calcium, preferably sodium, more preferably citric acid, succinic acid and/or di ethylene triamine penta acetic acid, most preferably citric acid and its salts, preferably its citric acid.

### Embodiment 15

Graft polymer according to any of Embodiments 9 to 14, wherein the molar ratio of the total amount of the metal salt and the total amount of the at least one organic compound is between 1:1 to 1:10,000, preferred 1:2 to 1:1,000, mostly preferred between 1:5 to 1:500, such as 1:10 to 1:400, 1:30 to 1:200, 1:50 to 1:200, or about 1:80 to 1:120.

### Embodiment 16

Graft polymer according to any of Embodiments 12 to 15 wherein the organic compounds are chelating agents which exhibit a “Ready Biodegradability” according to the OCED Guideline (Test No. 301).

### Embodiment 17

Graft polymer according any of the previous Embodiments in dried form, such as spray-dried, freeze-dried, vacuum-dried, roller drum-dried, or in granule or granulate form.

### Embodiment 18

30 Graft polymer according to any of Embodiments 1-16 in solution form, preferably as an aqueous solution, more preferably as a solution in essentially water.

### Embodiment 19

Graft polymer according to any of the previous Embodiments which fulfills at least one of the following requirements, preferably more than one, such as in the order of preference b> c> d> a, such as b+c, more preferably b+c+d, even more preferably all of them:

is readily biodegradable according to the tests as defined by OECD 301F and/or B guidelines, preferably exhibiting a biodegradation in weight percent based on the total polymer weight of at least 50% within 28 days according to OECD 301F and/or B (based on the total solid content), and/or

exhibits a Gardner color less than 4.0, and/or

exhibits a color stability at least 6 months with an increase of maximum three Gardner color value units, and/or and the invented polymer forms a clear liquid solution in the liquid detergent formulation, without any turbidity and precipitations in the liquid detergent formulation, at least 28 days by a minimum content of 0.5% of invented polymer in the liquid detergent's formulation.

### Embodiment 20

Process to produce a graft polymer according to any of the previous Embodiments, wherein as reactants
a polymer base being at least one polysaccharide, preferably the polysaccharide consisting of a maximum of up to 30 glycose units in average, more preferably consisting of maltodextrin, corn syrups and or corn syrups solids consisting of up to 20, preferably 2 to 20, more preferably 3 to 10 glycose units on average, and
at least one water soluble ethylenically unsaturated monomer, such unsaturated monomer comprising at least one sodium carboxylate unit—which may be neutralized from the carboxylic acid-form before adding to the reactor (i.e. when the polysaccharide is not present during the neutralization), in situ (in the reactor when the polysaccharide is present)—wherein preferably at least 50 mol %, preferably at least 90 mol % of the total amount of the monomer(s) employed for the polymerization reaction is sodium acrylate, and optionally at least one other polymerizable monomer, and most preferably the sodium acrylate is prepared before adding into the reaction mixture,

are polymerized by radically initiated polymerization reaction, preferably by radical redox polymerization-type reaction, more preferably using as radical initiators for at least a main polymerization stage a polymerization system comprising at least one metal salt and hydrogen peroxide, wherein the metal salt comprises iron, preferably contains iron as only metal, which is more preferably employed as ferrous sulfate heptahydrate, further adding before or during at least the main polymerization reaction at least one organic compound containing in its structure at least two carboxyl-groups which may be partially or fully present as acid-group or deprotonated in their salt-form, such organic compound being selected from the groups as defined in previous Embodiments 12 to 14, preferably Embodiments 13 to 14, more preferably Embodiment 14;

such polymerization reaction(s) taking place in a suitable solvent or solvent mixture, such solvent(s) being selected from polar protic or polar aprotic solvents, preferably acetone, ethanol, isopropanol or water, more preferably aqueous solutions, most preferably essentially only water, optionally adding in the post-polymerization step which follows after the main polymerization step a further portion of the same initiator or initiator system as employed in the main polymerization stage or a different initiator or initiator system, selected from azo initiators, redox initiators, peroxides, photo-initiators and mixtures thereof, preferably water soluble initiator systems, most preferably sodium persulfate and the combination of sodium persulfate with hydrogen peroxide, to further reduce the amount of unreacted monomers within the polymerization reaction mixture, to obtain a polymer solution comprising the graft polymer and the organic compound(s),

optionally subjection of the polymer solution to a further treatment selected from steam stripping, steam distillation, thermal distillation and/or vacuum distillation, to obtain a polymer solution which is purified compared to the polymer solution before this present step, and

optionally drying the polymer solution obtained in the previous step to obtain the graft polymer in solid form, by employing as drying means at least one spray-drier, freeze-drier, vacuum-drier, roller drum-drier, paddle drier, kneader, or any means for agglomeration such as fluidized bed-agglomeration and the like to obtain the polymer as agglomerates or granules or granulates, preferred are drying methods, which have a low temperature impact on the polymer to prevent discoloration, like spray-drier, freeze-drier, vacuum-drier, agglomeration such as fluidized bed-agglomeration, to obtain a polymer solid.

### Embodiment 21

Process according to Embodiment 20 wherein the polymerization takes place at a temperature of from 70 to 150, preferably 80 to 120, more preferably 90 to 100° C., and the post-polymerisation reaction takes place at a temperature that is lower by about 5 to 10 degree compared to the temperature of the main polymerization reaction.

### Embodiment 22

Process according to any of Embodiments 20 to 21 wherein the polymerization takes place at a solids concentration based on the combined total amount of polysaccharide and monomers employed of from 20 to 60, preferably 25 to 50 wt. %.

### Embodiment 23

Process according to any of Embodiments 20 to 22 wherein the graft polymer is produced by reacting an aqueous solution of corn syrup consisting of 3 to 8 glycose units on average with a mixture of acrylic acid and sodium acrylate with an degree of neutralization of this mixture of 90 to 96%, using a hydrogen peroxide initiator and ferrous sulfate heptahydrate redox catalyst, with the use of citric acid or their salts and preferably citric acid monohydrate or aqueous citric acid solution during the polymerization and post-polymerization to chelate metal ion(s), and using sodium persulfate in combination with hydrogen peroxide during post-polymerization to consume residual acrylate monomer,

with a polymerization temperature from 90 to 100° C. and a post polymerization temperature from 85 to 95° C., with a graft polymer solids content after the complete polymerization from 20 to 60 preferred 25 to 50 wt. %.

### Embodiment 24

Use of at least one graft polymer according to any of Embodiments 1 to 19 or obtained by or obtainable by the process according to any of Embodiments 20 to 23 in a composition, that is a cleaning composition, fabric and home care product, an industrial and institutional cleaning product, cosmetic or personal care product, or agrochemical formulations.

### Embodiment 25

The use according to Embodiment 24 in compositions for fabric and home care, cleaning composition, or an industrial and institutional cleaning product.

### Embodiment 26

The use according to Embodiment 24 or 25, wherein the composition comprises at least one graft polymer at a concentration of from about 0.05% to about 10% in weight % in relation to the total weight of such composition or product.

### Embodiment 27

The use according to any of Embodiments 24 to 26, comprising at least one enzyme, and/or

about 1% to about 70% by weight of a surfactant system, and/or at least one further cleaning adjunct in effective amounts, and/or exhibiting an improved washing performance in red clay dispersion in comparison to sodium polyacrylate-polymer having a Fikentscher's K-Value of 15 when substituted by the graft polymer within such composition.

### Embodiment 28

A laundry detergent composition, a cleaning composition or a fabric and home care product, preferably a laundry detergent composition, more preferably a liquid or semi-liquid or multi-component laundry detergent composition, most preferably a liquid laundry detergent composition, comprising at least one graft polymer according to any of Embodiments 1 to 19 or obtained by or obtainable by the process according to any of Embodiments 20 to 23, comprising the at least one graft polymer at a concentration of preferably from about 0.05% to about 10% in weight % in relation to the total weight of such composition or product, and optionally further comprising

at least one enzyme, and/or

about 1% to about 70% by weight of a surfactant system, and/or

at least one further cleaning adjunct in effective amounts.

### Embodiment 29

A dish wash detergent composition, preferably a liquid dish wash detergent composition or a solid automated dish wash detergent composition, more preferably a liquid hand dish wash detergent composition, comprising at least one graft polymer Embodiments 1 to 19 or obtained by or obtainable by the process according to any of Embodiments 20 to 23, comprising the at least one graft polymer at a concentration of from about 0.05% to about 10% in weight % in relation to the total weight of such composition, and optionally further comprising

at least one enzyme, and/or

about 1% to about 70% by weight of a surfactant system, and/or

at least one further cleaning adjunct in effective amounts, and/or

at least one chelant, and/or

at least one anionic surfactant.

### Embodiment 30

Method of stabilizing a graft polymer according to any of Embodiments 1 to 19 or obtained by or obtainable by the process according to any of Embodiments 20 to 23 by adding at least one organic compound as defined in any of Embodiments 12 to 14 by adding it to the polymerized graft polymer at a molar ratio of the total amount of the at least one organic compound and the total amount of the graft polymer in between 1:100 to 1:1 Mio, preferred 1:500 to 1:100,000, and most preferred between 1:1,000 to 1:10,000 (including the limits given).

Use of and compositions comprising at least one graft polymer Part of this invention is also the use of the inventive polymer for various fields of applications, where they can replace currently known similar structures, but bring in their enhanced rate of biodegradation compared to those previously known structures.

### Embodiment 31

Use of at least one polymer according to any one of Embodiments 1 to 19 or obtained by or obtainable by the process according to any of Embodiments 20 to 23 in
a) cleaning compositions, preferably as additive for liquid, solid or semi-solid detergent formulations, particularly for liquid detergent formulations, preferably concentrated liquid detergent formulations or single mono doses laundry detergent formulations, or liquid hand dish washing detergent formulations or solid automatic dish washing formulations;
b) in fabric and home care products,
c) in formulations for electro plating;
d) in cementitious compositions;
e) in agrochemical formulations, preferably as dispersant;
f) as adhesion promoters, for example for printing inks for laminate films;
g) as an assistant (adhesion), for example for production of multilayer composite films, with compatibilization not just of different polymer layers but also of metal foils;
h) as adhesion promoters for adhesives, for example in conjunction with polyvinyl alcohol, butyrate and acetate and styrene copolymers, or as a cohesion promoter for label adhesives;
i) as a primer in coatings applications for improvement of adhesion on substrates such as glass, wood, plastic and metal;
j) for improvement of wet adhesion, for example in standard emulsion paints, and for improvement of instantaneous rain resistance of paints, for example for road markings;
k) as complexing agents, especially with high binding capacity for heavy metals such as Hg, Pb, Cu, Ni;
l) as a flocculant, for example in water treatment/water processing;
m) as a penetration aid, for example for active metal salt formulations in wood protection;
n) as corrosion inhibitors, for example for iron and nonferrous metals, and in the sectors of petroleum production and of secondary oil production;
o) for immobilization of proteins and enzymes; microorganisms or as immobilizing supports of enzymes and microorganisms;
p) for blocking and sealing, for example mineral oil and natural gas industry;
q) as fixatives, for example in the textile industry, especially as formaldehyde-free cofixers;
r) as an additive in the cosmetic formulations, for example for hair-setting compositions and hair rinses;
s) as an assistant in the papermaking industry, for example for acceleration of dewatering, elimination of contraries, neutralization of charge and paper coating as a multipurpose assistant;
t) for separation of oil and water, for example in the metalworking industry;
u) as an additive for landfill seals;
v) as a flocculant;
w) as a swimming pool algicide;
x) for production of bitumen chemicals by reaction with fatty acids;
y) as an antiswelling agent in order that clay absorbs water in a retarded manner; z) as an emulsifier or emulsion breaker;
aa) as a surfactant in the industrial cleaning (IC) sector;
bb) as a wood protector;
cc) for preparation of complexing agents (polycarboxylates);
dd) for production of assistants for ore mining and mineral processing;
ee) as a dispersant for pigments, ceramic, carbon black, carbon, carbon fibers, metal powders, such as emulsifier or dispersant for inks for e.g. ink jet printing;
ff) for gas scrubbing as an absorbent of CO2, NOX, SOX, Cl2 and aldehydes, and for neutralization of acidic constituents;
gg) for water softening;
hh) as a crystallization inhibitor in e.g. agrochemical formulations, oil-field uses;
ii) as a rheology modifier (thickener);
jj) as an assistant or as a component for assistants for the extraction and processing of oil, coal and natural gas;
kk) for production of synthetic rubber and rubber chemicals;
ll) as an additive in coolants, lubricants and cooling lubricants;
mm) as assistants in the construction chemicals sector;
nn) as a constituent of galvanizing baths; or
oo) for production of nonviral gene vectors.

A subject matter of the present invention is the use of the above-mentioned polymer (including the modified polymer) in fabric and home care products, in cosmetic formulations, as crude oil emulsion breaker, in pigment dispersions for ink jet inks, in formulations for electro plating, in cementitious compositions and/or as dispersant for agrochemical formulations, preferably in cleaning compositions and/or in fabric and home care products, in particular cleaning compositions for improved oily and fatty stain removal, wherein the cleaning composition is preferably a laundry detergent formulation and/or a manual dish wash detergent formulation, more preferably a liquid laundry detergent formulation and/or a liquid manual dish wash detergent formulation.

The polymer can be added to cosmetic formulations, as crude oil emulsion breaker, in pigment dispersions for ink jet inks, formulations for electro plating, in cementitious compositions.

However, the inventive compounds can also be added to (used in) washing or cleaning compositions.

Another subject-matter of the present invention is, therefore, a cleaning composition, fabric and home care product, industrial and institutional cleaning product, cosmetic formulation, crude oil emulsion breaker, pigment dispersion for ink jet inks, formulation for electro plating, cementitious composition and/or dispersant for agrochemical formulations, comprising at least one polymer, as defined above.

Preferably, it is a cleaning composition and/or fabric and home care product, comprising at least one polymer, as defined above, preferably for improved oily and fatty stain removal, preferably a laundry detergent formulation and/or a manual dish wash detergent formulation, more preferably a liquid laundry detergent formulation and/or a liquid manual dish wash detergent formulation.

In another preferred embodiment of the present invention, the cleaning composition may be used for soil removal of particulate stains and/or oily and fatty stains, and additionally for whiteness maintenance, preferably in laundry care.

In another embodiment, the cleaning composition of the present invention is a hard surface cleaning composition that may be used for cleaning various surfaces such as hard wood, tile, ceramic, plastic, leather, metal, glass.

In another embodiment, the cleaning composition of the present invention is a liquid or solid automatic dish wash detergent composition, preferably a solid automatic dish wash detergent composition, that may be used for cleaning dish ware, e.g., dish ware such as glasses, wherein the inventive polymer is preventing the corrosion of glass surfaces.

In another embodiment, the cleaning composition is designed to be used in personal care and pet care compositions such as shampoo compositions, body wash formulations, liquid or solid soaps.

In this invention, a preferred area of application for the use of the polymer is the field of fabric and home care products and cleaning compositions, preferably cleaning compositions for industrial and institutional use and the use by consumers in their household.

### Embodiment 32

The use according to Embodiment 31 in cleaning compositions and/or in fabric and home care products, preferably in liquid and solid detergent compositions, such detergent compositions preferably being
a) manual and automatic dish wash detergent compositions, comprising the at least one polymer, and the at least one chelating agent and/or the at least one surfactant or—more preferably—a chelating agent in case of a liquid or solid automatic dish wash composition and a surfactant system in case of a liquid manual dish wash detergent composition, respectively; and/or
b) laundry detergent compositions comprising the at least one polymer, and at least one surfactant or—preferably—a surfactant system.

Within such preferred application areas of use, typical tasks have to be fulfilled, all of which are commonly encompassed by the term “cleaning”, but in fact comprise different tasks such as removing oily and fatty residues, solid residues, amphiphilic residues and hydrophilic residues.

Other tasks are the protection of the goods to be cleaned from deterioration, such as protecting glass from corroding, silverware from oxidation, colours from fading etc. Other tasks are improving the overall appearance of the to be cleaned goods, such as increasing or restoring the colour, the whiteness, imparting or increasing a shine. For many such applications additional ingredients are typically added, for cleaning applications important ones are for example enzymes, which help biologically to degrade residues.

### Embodiment 33

The use according to Embodiment 32 for
i) improved removal of oily/fatty stains, and/or
ii) clay removal, and/or
iii) soil removal of particulate stains, and/or
iv) dispersion and/or emulsification of soils, and/or
v) modification of treated surface to improve removal upon later re-soiling, and/or
vi) prevention or reduction, preferably prevention, of glass corrosion, and/or
vii) whiteness improvement, and/or
viii)—when at least one enzyme selected from the list consisting of lipases, hydrolases, amylases, proteases, cellulases, hemicellulases, phospholipases, esterases, pectinases, lactases and peroxidases, and combinations of at least two of the foregoing types, is present—additionally for improvement of removal of oily/fatty stains, food stain removal and/or removal of complex stains, most preferably in cleaning compositions for i) Improved removal of oily/fatty stains, each of the before mentioned options i) to viii) preferably for use in a laundry detergent formulation and/or a dish wash detergent formulation, more preferably in a liquid laundry detergent formulation, and/or an—preferably solid—automatic dish wash detergent or a liquid manual dish wash detergent formulation.

### Embodiment 34

The use according to any of Embodiments 32 to 33 in cleaning compositions and/or in fabric and home care products, preferably in cleaning compositions for in fabric and home care, the cleaning composition preferably being a laundry detergent formulation or a dish wash detergent formulation.

Such ingredients are typically formulated with other ingredients in formulations and compositions, which may be also called “products” (as they are provided from a supplier as a formulation to another customer who uses such formulation directly for cleaning purposes etc. or for producing another formulation, which in turn could be sold to consumers as a “product” to be used by the consumer.

### Embodiment 35

A composition that is a fabric and home care product, cleaning composition, industrial and institutional cleaning product, cosmetic or personal care product, oil field-formulation such as crude oil emulsion breaker, pigment dispersion for inks such as ink-jet inks, electro plating product, cementitious composition, lacquer, paint, agrochemical formulation, preferably a laundry detergent, a dish wash composition, a cleaning composition and/or a fabric and home care product, each comprising at least one graft polymer according to any of the Embodiments 1 to 19 or obtained by or obtainable by a process according to any of Embodiments 20-23.

### Embodiment 36

A composition according to Embodiment 35 being a solid or liquid laundry detergent composition or a solid or liquid manual dish wash detergent composition, preferably a liquid laundry detergent or manual dish wash detergent composition, more preferably a liquid laundry detergent composition, comprising the least one polymer, preferably the at least one graft polymer according to any one of Embodiments 5 to 15; optionally further comprising at least one enzyme, preferably selected from one or more lipases, hydrolases, amylases, proteases, cellulases, hemicellulases, phospholipases, esterases, pectinases, lactases, pectate lyases, mannanases and peroxidases, and combinations of at least two of the foregoing types, preferably at least one enzyme being selected from proteases,
optionally containing at least one antimicrobial agent, wherein the at least one polymer is present in an amount ranging from about 0.01% to about 20%, preferably from about 0.05% to 15%, more preferably from about 0.1% to about 10%, and most preferably from about 0.5% to about 5%, in relation to the total weight of such composition or product, and

such product or composition further comprising from about 1% to about 70% by weight of at least one surfactant, preferably an anionic surfactant, or even more preferably of a surfactant system comprising at least one anionic surfactant.

### Embodiment 37

A composition according to Embodiment 35 being a solid or liquid automatic dish wash detergent composition, preferably a solid automatic dish wash detergent composition, comprising the at least one graft polymer,

optionally further comprising at least one enzyme, preferably selected from one or more lipases, hydrolases, amylases, proteases, cellulases, hemicellulases, phospholipases, esterases, pectinases, lactases, pectate lyases, mannanases and peroxidases, and combinations of at least two of the foregoing types, preferably at least one enzyme being selected from proteases and amylases,

optionally containing at least one antimicrobial agent,

optionally containing at least one compound selected from alkali metal percarbonate, alkali metal perborate and alkali metal persulfate, optionally containing at least one zinc salt,

wherein the at least one graft polymer is being present in a total amount ranging from about 0.001% to about 10%, preferably from about 0.005% to 5%, more preferably from about 0.01% to about 3%, and most preferably from about 0.1% to about 2%, and such product or composition further comprising at least one chelating agent being present in a total amount from about 1% to about 70%, preferably from 10% to about 60% and even more preferably from 30% to about 50%, and

optionally further comprising at least one surfactant or more preferably a surfactant system in a total amount of from about 1% to about 70% by weight, all weight percent in relation to the total weight of such composition.

### Embodiment 38

A composition according to Embodiment 37, being a solid automatic dish wash detergent composition, comprising the at least one graft polymer, and

additionally comprising at least one chelating agent selected from methylglycinediaceticacid (MGDA), glutamic acid diacetate (GLDA), citric acid and salts thereof,

at least one enzyme selected from proteases and/or amylases,

at least one bleaching agent selected from alkali metal percarbonate, alkali metal perborate and alkali metal persulfate, preferably alkali metal percarbonate,

at least one non-ionic surfactant,

optionally at least one disintegrant, preferably a super-disintergrant, more preferably PVPP, and optionally containing at least one zinc salt.

Super-disintegrants are known by a person of skill in the art, e.g. from EP1004661, EP1263814 and EP1036839, and are discussed also in Pharmaceutical Technology, Volume 2006 Supplement, Issue 5, “A Comparative Study of Current Superdisintegrants”, Oct. 1, 2006.

### Embodiment 39

Composition according to any of Embodiments 35 to 36 being a detergent composition, comprising as surfactant at least one anionic surfactant, and further comprising water.

### Embodiment 40

Composition according to any of Embodiments 37 and 38 being a detergent composition, comprising as surfactant at least one non-ionic surfactant, and further comprising water.

### Embodiment 41

Composition according to any of Embodiments 35 to 40 being a detergent composition, comprising at least one further polymer selected from multifunctional polyethylene imines or multifunctional diamines, or mixtures thereof.

### Embodiment 42

Composition according to any of Embodiments 35 to 41 being a liquid detergent composition, comprising as surfactant at least one 2-propylheptyl ethoxylated non-ionic surfactant having an average degree of ethoxylation of from 3 to 5.

Further, the invention may be characterized further by the following: It is also preferred in the present invention that the cleaning composition comprises (besides at least one polymer as described above) additionally at least one enzyme, preferably selected from one or more lipases, hydrolases, amylases, proteases, cellulases, hemicellulases, phospholipases, esterases, pectinases, lactases and peroxidases, and combinations of at least two of the foregoing types.

Preferably, the such inventive cleaning composition is a fabric and home care product or an industrial and institutional (l&l) cleaning product, preferably a fabric and home care product, more preferably a laundry detergent or manual dish washing detergent, comprising at least one inventive polymer, and optionally further comprising at least one surfactant or a surfactant system, providing improved removal, dispersion and/or emulsification of soils and/or modification of treated surfaces and/or whiteness maintenance of treated surfaces.

At least one inventive polymer as described herein (such polymer as defined before and especially in the Embodiments 1 to 19 or obtained or obtainable by a process according to any of Embodiments 20 to 23 are in this following section also termed “inventive polymer”) is present in said inventive cleaning compositions at a concentration of 0.001 to 10, preferably from about 0.005% to 5%, more preferably from about 0.01% to about 5%, and most preferably from about 0.1% to about 3%, in relation to the total weight of such composition; such cleaning composition may—and preferably does—further comprise a from about 1% to about 70% by weight of a surfactant system.

Even more preferably, the cleaning compositions of the present invention comprising at least one inventive polymer, and optionally further comprising at least one surfactant or a surfactant system, are those for primary cleaning (i.e., removal of stains) within laundry and manual dish wash applications, even more specifically, for removal of oily and fatty stains such as those on fabrics and dishware, and may additionally comprise at least one enzyme selected from the list consisting of lipases, hydrolases, amylases, proteases, cellulases, hemicellulases, phospholipases, esterases, pectinases, lactases and peroxidases, and combinations of at least two of the foregoing types of enzymes.

In one preferred embodiment, the cleaning composition of the present invention is a liquid or solid laundry detergent composition.

In another preferred embodiment, the cleaning composition of the present invention is a liquid or solid (e.g., powder or tab/unit dose) detergent composition for manual or automatic dish wash, preferably either a liquid manual dish wash detergent composition or a solid automatic dish wash composition.

In one embodiment, the inventive polymers of the present invention may be utilized in cleaning compositions comprising a surfactant system comprising C10-C15 alkyl benzene sulfonates (LAS) as the primary surfactant and one or more additional surfactants selected from non-ionic, cationic, amphoteric, zwitterionic or other anionic surfactants, or mixtures thereof.

In a further embodiment, the inventive polymers may be utilized in cleaning compositions, such as laundry detergents of any kind, and the like, comprising C8-C18 linear or branched alkyl ethersulfates with 1-5 ethoxy-units as the primary surfactant and one or more additional surfactants selected from non-ionic, cationic, amphoteric, zwitterionic or other anionic surfactants, or mixtures thereof.

In a further embodiment the inventive polymers may be utilized in cleaning compositions, such as laundry detergents of any kind, and the like, comprising C12-C18 alkyl ethoxylate surfactants with 5-10 ethoxy-units as the primary surfactant and one or more additional surfactants selected from anionic, cationic, amphoteric, zwitterionic or other non-ionic surfactants, or mixtures thereof.

In one embodiment of the present invention, the inventive polymer is a component of a cleaning composition, such as preferably a laundry or a dish wash formulation, more preferably a liquid laundry or manual dish wash formulation, that each additionally comprise at least one surfactant, preferably at least one anionic surfactant.

The selection of the additional surfactants in these embodiments may be dependent upon the application and the desired benefit.

Description of Cleaning Compositions, Formulations and their Ingredients

The phrase “cleaning composition” as used herein includes compositions and formulations designed for cleaning soiled material. Such compositions and formulations include those designed for cleaning soiled material or surfaces of any kind.

Compositions for “industrial and institutional cleaning” includes such cleaning compositions being designed for use in industrial and institutional cleaning, such as those for use of cleaning soiled material or surfaces of any kind, such as hard surface cleaners for surfaces of any kind, including tiles, carpets, PVC-surfaces, wooden surfaces, metal surfaces, lacquered surfaces.

“Compositions for Fabric and Home Care” include cleaning compositions including but not limited to laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, dish washing compositions, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation, preferably during the wash cycle of the laundering or dish washing operation.

The cleaning compositions of the invention may be in any form, namely, in the form of a liquid; a solid such as a powder, granules, agglomerate, paste, tablet, pouches, bar, gel; an emulsion; types delivered in dual- or multi-compartment containers; single-phase or multi-phase unit dose; a spray or foam detergent; premoistened wipes (i.e., the cleaning composition in combination with a nonwoven material such as that discussed in U.S. Pat. No. 6,121,165, Mackey, et al.); dry wipes (i.e., the cleaning composition in combination with a nonwoven materials, such as that discussed in U.S. Pat. No. 5,980,931, Fowler, et al.) activated with water by a user or consumer; and other homogeneous, non-homogeneous or single-phase or multiphase cleaning product forms.

The liquid cleaning compositions of the present invention preferably have a viscosity of from 50 to 10,000 mPa*s; liquid manual dish wash cleaning compositions (also liquid manual “dish wash compositions”) have a viscosity of preferably from 100 to 10,000 mPa*s, more preferably from 200 to 5,000 mPa*s and most preferably from 500 to 3,000 mPa*s at 20 1/s and 20° C.; liquid laundry cleaning compositions have a viscosity of preferably from 50 to 3,000 mPa*s, more preferably from 100 to 1,500 mPa*s and most preferably from 200 to 1,000 mPa*s at 20 1/s and 20° C.

The liquid cleaning compositions of the present invention may have any suitable pH-value. Preferably the pH of the composition is adjusted to between 4 and 14. More preferably the composition has a pH of from 6 to 13, even more preferably from 6 to 10, most preferably from 7 to 9. The pH of the composition can be adjusted using pH modifying ingredients known in the art and is measured as a 10% product concentration in demineralized water at 25° C. For example, NaOH may be used and the actual weight % of NaOH may be varied and trimmed up to the desired pH such as pH 8.0. In one embodiment of the present invention, a pH>7 is adjusted by using amines, preferably alkanolamines, more preferably triethanolamine.

Cleaning compositions such as fabric and home care products and formulations for industrial and institutional cleaning, more specifically such as laundry and manual dish wash detergents, are known to a person skilled in the art. Any composition etc. known to a person skilled in the art, in connection with the respective use, can be employed within the context of the present invention by including at least one inventive polymer, preferably at least one polymer in amounts suitable for expressing a certain property within such a composition, especially when such a composition is used in its area of use.

One aspect of the present invention is also the use of the inventive polymers as additives for detergent formulations, particularly for liquid detergent formulations, preferably concentrated liquid detergent formulations, or single mono doses for laundry.

The cleaning compositions of the invention may—and preferably do—contain adjunct cleaning additives (also abbreviated herein as “adjuncts”), such adjuncts being preferably in addition to a surfactant system as defined before.

Suitable adjunct cleaning additives include builders, cobuilders, structurants or thickeners, clay soil removal/anti-redeposition agents, polymeric soil release agents, dispersants such as polymeric dispersing agents, polymeric grease cleaning agents, solubilizing agents, chelating agents, enzymes, enzyme stabilizing systems, bleaching compounds, bleaching agents, bleach activators, bleach catalysts, brighteners, malodor control agents, pigments, dyes, opacifiers, hueing agents, dye transfer inhibiting agents, chelating agents, suds boosters, suds suppressors (antifoams), color speckles, silver care, anti-tarnish and/or anti-corrosion agents, alkalinity sources, pH adjusters, pH-buffer agents, hydrotropes, scrubbing particles, antibacterial agents, anti-oxidants, softeners, carriers, processing aids, pro-perfumes, and perfumes.

Liquid cleaning compositions additionally may comprise—and preferably do comprise at least one of—rheology control/modifying agents, emollients, humectants, skin rejuvenating actives, and solvents.

Solid compositions additionally may comprise—and preferably do comprise at least one of—fillers, bleaches, bleach activators and catalytic materials.

Suitable examples of such cleaning adjuncts and levels of use are found in WO 99/05242, U.S. Pat. Nos. 5,576,282, 6,306,812 B1 and 6,326,348 B1.

Those of ordinary skill in the art will understand that a detersive surfactant encompasses any surfactant or mixture of surfactants that provide cleaning, stain removing, or laundering benefit to soiled material.

Hence, the cleaning compositions of the invention such as fabric and home care products, and formulations for industrial and institutional cleaning, more specifically such as laundry and manual dish wash detergents, preferably additionally comprise a surfactant system and, more preferably, also further adjuncts, as the one described above and below in more detail.

The surfactant system may be composed from one surfactant or from a combination of surfactants selected from anionic surfactants, non-ionic surfactants, cationic surfactants, zwitterionic surfactants, amphoteric surfactants, and mixtures thereof. Those of ordinary skill in the art will understand that a surfactant system for detergents encompasses any surfactant or mixture of surfactants that provide cleaning, stain removing, or laundering benefit to soiled material.

The cleaning compositions of the invention preferably comprise a surfactant system in an amount sufficient to provide desired cleaning properties. In some embodiments, the cleaning composition comprises, by weight of the composition, from about 1% to about 70% of a surfactant system. In other embodiments, the liquid cleaning composition comprises, by weight of the composition, from about 2% to about 60% of the surfactant system. In further embodiments, the cleaning composition comprises, by weight of the composition, from about 5% to about 30% of the surfactant system. The surfactant system may comprise a detersive surfactant selected from anionic surfactants, non-ionic surfactants, cationic surfactants, zwitterionic surfactants, amphoteric surfactants, and mixtures thereof.

### (a) Laundry Compositions

In laundry formulations, anionic surfactants contribute usually by far the largest share of surfactants within such formulation. Hence, preferably, the inventive cleaning compositions for use in laundry comprise at least one anionic surfactant and optionally further surfactants selected from any of the surfactant classes described herein, preferably from non-ionic surfactants and/or amphoteric surfactants and/or zwitterionic surfactants and/or cationic surfactants.

Nonlimiting examples of anionic surfactants—which may be employed also in combinations of more than one surfactant—useful herein include C9-C20 linear alkylbenzenesulfonates (LAS), C10-C20 primary, branched chain and random alkyl sulfates (AS); C10-C18 secondary (2,3)alkyl sulfates; C10-C18 alkyl alkoxy sulfates (AExS) wherein x is from 1 to 30; C10-C18 alkyl alkoxy carboxylates comprising 1 to 5 ethoxy units; mid-chain branched alkyl sulfates as discussed in U.S. Pat. Nos. 6,020,303 and 6,060,443; mid-chain branched alkyl alkoxy sulfates as discussed in U.S. Pat. Nos. 6,008,181 and 6,020,303; modified alkylbenzene sulfonate (MLAS) as discussed in WO 99/05243, WO 99/05242 and WO 99/05244; methyl ester sulfonate (MES); and alpha-olefin sulfonate (AOS).

Preferred examples of suitable anionic surfactants are alkali metal and ammonium salts of C8-C12-alkyl sulfates, of C12-C18-fatty alcohol ether sulfates, of C12-C18-fatty alcohol polyether sulfates, of sulfuric acid half-esters of ethoxylated C4-C12-alkylphenols (ethoxylation: 3 to 50 mol of ethylene oxide/mol), of C12-C18-alkylsulfonic acids, of C12-C18 sulfo fatty acid alkyl esters, for example of C12-C18 sulfo fatty acid methyl esters, of C10-C18-alkylarylsulfonic acids, preferably of n-C10-C18-alkylbenzene sulfonic acids, of C10-C18 alkyl alkoxy carboxylates and of soaps such as for example C8-C24-carboxylic acids. Preference is given to the alkali metal salts of the aforementioned compounds, particularly preferably the sodium salts.

In one embodiment of the present invention, anionic surfactants are selected from n-C10-C18-alkylbenzene sulfonic acids and from fatty alcohol polyether sulfates, which, within the context of the present invention, are in particular sulfuric acid half-esters of ethoxylated C12-C18-alkanols (ethoxylation: 1 to 50 mol of ethylene oxide/mol), preferably of n-C12-C18-alkanols.

In one embodiment of the present invention, also alcohol polyether sulfates derived from branched (i.e., synthetic) C11-C18-alkanols (ethoxylation: 1 to 50 mol of ethylene oxide/mol) may be employed.

Preferably, the alkoxylation group of both types of alkoxylated alkyl sulfates, based on C12-C18-fatty alcohols or based on branched (i.e., synthetic) C11-C18-alcohols, is an ethoxylation group and an average ethoxylation degree of any of the alkoxylated alkyl sulfates is 1 to 5, preferably 1 to 3.

Preferably, the laundry detergent formulation of the present invention comprises from at least 1 wt.-% to 50 wt.-%, preferably in the range from greater than or equal to about 2 wt.-% to equal to or less than about 30 wt.-%, more preferably in the range from greater than or equal to 3 wt.-% to less than or equal to 25 wt.-%, and most preferably in the range from greater than or equal to 5 wt.-% to less than or equal to 25 wt.-% of one or more anionic surfactants as described above, based on the particular overall composition, including other components and water and/or solvents.

In a preferred embodiment of the present invention, anionic surfactants are selected from C10-C15 linear alkylbenzenesulfonates, C10-C18 alkylethersulfates with 1-5 ethoxy units and C10-C18 alkylsulfates.

Non-limiting examples of non-ionic surfactants—which may be employed also in combinations of more than one other surfactant—include: C8-C18 alkyl ethoxylates, such as, NEODOL® non-ionic surfactants from Shell; ethylenoxide/propylenoxide block alkoxylates as PLURONIC® from BASF; C14-C22 mid-chain branched alkyl alkoxylates, BAEx, wherein x is from 1 to 30, as discussed in U.S. Pat. Nos. 6,153,577, 6,020,303 and 6,093,856; alkylpolysaccharides as discussed in U.S. Pat. No. 4,565,647 Llenado, issued Jan. 26, 1986; specifically alkylpolyglycosides as discussed in U.S. Pat. Nos. 4,483,780 and 4,483,779; polyhydroxy fatty acid amides as discussed in U.S. Pat. No. 5,332,528; and ether capped poly(oxyalkylated) alcohol surfactants as discussed in U.S. Pat. No. 6,482,994 and WO 01/42408.

Preferred examples of non-ionic surfactants are in particular alkoxylated alcohols and alkoxylated fatty alcohols, di- and multiblock copolymers of ethylene oxide and propylene oxide and reaction products of sorbitan with ethylene oxide or propylene oxide, furthermore alkylphenol ethoxylates, alkyl glycosides, polyhydroxy fatty acid amides (glucamides). Examples of (additional) amphoteric surfactants are so-called amine oxides.

Preferred examples of alkoxylated alcohols and alkoxylated fatty alcohols are, for example, compounds of the general formula (A)

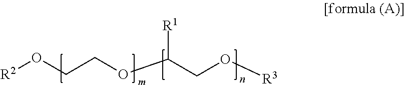

in which the variables are defined as follows:
R1 is selected from linear C1-C10-alkyl, preferably ethyl and particularly preferably methyl,
R2 is selected from C8-C22-alkyl, for example n-C8H17, n-C10H21, n-C12H25, n-C14H29, n-C16H33 or n-C18H37,
R3 is selected from C1-C10-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl, m and n are in the range from zero to 300, where the sum of n and m is at least one.

Preferably, m is in the range from 1 to 100 and n is in the range from 0 to 30.

Here, compounds of the general formula (A) may be block copolymers or random copolymers, preference being given to block copolymers.

Other preferred examples of alkoxylated alcohols and alkoxylated fatty alcohols are, for example, compounds of the general formula (B)

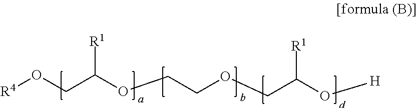

in which the variables are defined as follows:
R1 is identical or different and selected from linear C₁-C₄-alkyl, preferably identical in each case and ethyl and particularly preferably methyl,
R4 is selected from C6-C20-alkyl, in particular n-C8H17, n-C10H21, n-C12H25, n-C14H29, n-C16H33, n-C18H37,
a is a number in the range from zero to 6, preferably 1 to 6,
b is a number in the range from zero to 20, preferably 4 to 20,
d is a number in the range from 4 to 25.

Preferably, at least one of a and b is greater than zero.

Here, compounds of the general formula (B) may be block copolymers or random copolymers, preference being given to block copolymers.

Further suitable non-ionic surfactants are selected from di- and multiblock copolymers, composed of ethylene oxide and propylene oxide. Further suitable non-ionic surfactants are selected from ethoxylated or propoxylated sorbitan esters. Alkylphenol ethoxylates or alkyl polyglycosides or polyhydroxy fatty acid amides (glucamides) are likewise suitable. An overview of suitable further non-ionic surfactants can be found in EP-A 0 851 023 and in DE-A 198 19 187.

Mixtures of two or more different non-ionic surfactants may of course also be present.

In a preferred embodiment of the present invention, non-ionic surfactants are selected from C12/14 and C16/18 fatty alkoholalkoxylates, C13/15 oxoalkoholalkoxylates, C13-alkoholalkoxylates, and 2-propylheptylalkoholalkoxylates, each of them with 3-15 ethoxy units, preferably 5-10 ethoxy units, or with 1-3 propoxy- and 2-15 ethoxy units.

Non-limiting examples of amphoteric surfactants—which may be employed also in combinations of more than one other surfactant—include: water-soluble amine oxides containing one alkyl moiety of from about 8 to about 18 carbon atoms and 2 moieties selected from the group consisting of alkyl moieties and hydroxyalkyl moieties containing from about 1 to about 3 carbon atoms; and water-soluble sulfoxides containing one alkyl moiety of from about 10 to about 18 carbon atoms and a moiety selected from the group consisting of alkyl moieties and hydroxyalkyl moieties of from about 1 to about 3 carbon atoms. See WO 01/32816, U.S. Pat. Nos. 4,681,704; 4,133,779. Suitable surfactants include thus so-called amine oxides, such as lauryl dimethyl amine oxide (“lauramine oxide”).

Preferred examples of amphoteric surfactants are amine oxides. Preferred amine oxides are alkyl dimethyl amine oxides or alkyl amido propyl dimethyl amine oxides, more preferably alkyl dimethyl amine oxides and especially coco dimethyl amino oxides. Amine oxides may have a linear or mid-branched alkyl moiety. Typical linear amine oxides include water-soluble amine oxides containing one R1=C8-18 alkyl moiety and two R2 and R3 moieties selected from the group consisting of C₁-C₃ alkyl groups and C₁-C₃ hydroxyalkyl groups. Preferably, the amine oxide is characterized by the formula

R1—N(R2)(R3)—O

wherein R1 is a C8-18 alkyl and R2 and R3 are selected from the group consisting of methyl, ethyl, propyl, isopropyl, 2-hydroxethyl, 2-hydroxypropyl and 3-hydroxypropyl. The linear amine oxide surfactants in particular may include linear C10-C18 alkyl dimethyl amine oxides and linear C8-C12 alkoxy ethyl dihydroxy ethyl amine oxides. Preferred amine oxides include linear C10, linear C10-C12, and linear C12-C14 alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the alpha carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from 10 to 16. The number of carbon atoms for the one alkyl moiety (n1) should be approximately the same number of carbon atoms as the one alkyl branch (n2) such that the one alkyl moiety and the one alkyl branch are symmetric. As used herein “symmetric” means that (n1-n2) is less than or equal to 5, preferably 4, most preferably from 0 to 4 carbon atoms in at least 50 wt.-%, more preferably at least 75 wt.-% to 100 wt.-% of the mid-branched amine oxides for use herein. The amine oxide further comprises two moieties, independently selected from a C₁-C₃ alkyl, a C₁-C₃ hydroxyalkyl group, or a polyethylene oxide group containing an average of from about 1 to about 3 ethylene oxide groups. Preferably the two moieties are selected from a C₁-C₃ alkyl, more preferably both are selected as a C1 alkyl.

In a preferred embodiment of the present invention, amphoteric surfactants are selected from C8-C18 alkyl-dimethyl aminoxides and C8-C18 alkyl-di(hydroxyethyl)aminoxide.

Cleaning compositions may also contain zwitterionic surfactants—which may be employed also in combinations of more than one other surfactant.

Suitable zwitterionic surfactants include betaines, such as alkyl betaines, alkylamidobetaine, amidazoliniumbetaine, sulfobetaine (INCI Sultaines) as well as the phosphobetaines. Examples of suitable betaines and sulfobetaines are the following (designated in accordance with INCI): Almond amidopropyl of betaines, Apricotamidopropyl betaines, Avocadamidopropyl of betaines, Babassuamidopropyl of betaines, Behenamidopropyl betaines, Behenyl of betaines, Canol amidopropyl betaines, Capryl/Capramidopropyl betaines, Carnitine, Cetyl of betaines, Cocamidoethyl of betaines, Cocamidopropyl betaines, Cocamidopropyl Hydroxysultaine, Coco betaines, Coco Hydroxysultaine, Coco/Oleam idopropyl betaines, Coco Sultaine, Decyl of betaines, Dihydroxyethyl Oleyl Glycinate, Dihydroxyethyl Soy Glycinate, Dihydroxyethyl Stearyl Glycinate, Dihydroxyethyl Tallow Glycinate, Dimethicone Propyl of PG-betaines, Erucamidopropyl Hydroxysultaine, Hydrogenated Tallow of betaines, Isostearamidopropyl betaines, Lauramidopropyl betaines, Lauryl of betaines, Lauryl Hydroxysultaine, Lauryl Sultaine, Milkamidopropyl betaines, Minkamidopropyl of betaines, Myristamidopropyl betaines, Myristyl of betaines, Oleamidopropyl betaines, Oleamidopropyl Hydroxysultaine, Oleyl of betaines, Olivamidopropyl of betaines, Palmamidopropyl betaines, Palmitamidopropyl betaines, Palmitoyl Camitine, Palm Kemelamidopropyl betaines, Polytetrafluoroethylene Acetoxypropyl of betaines, Ricinoleam idopropyl betaines, Sesamidopropyl betaines, Soyamidopropyl betaines, Stearamidopropyl betaines, Stearyl of betaines, Tallowamidopropyl betaines, Tallowamidopropyl Hydroxysultaine, Tallow of betaines, Tallow Dihydroxyethyl of betaines, Undecylenamidopropyl betaines and Wheat Germamidopropyl betaines.

Preferred betaines are, for example, C12-C18-alkylbetaines and sulfobetaines. The zwitterionic surfactant preferably is a betaine surfactant, more preferable a Cocoamidopropylbetaine surfactant.

Non-limiting examples of cationic surfactants—which may be employed also in combinations of more than one other surfactant—include: the quaternary ammonium surfactants, which can have up to 26 carbon atoms include: alkoxylated quatemary ammonium (AQA) surfactants as discussed in U.S. Pat. No. 6,136,769; dimethyl hydroxyethyl quatemary ammonium as discussed in U.S. Pat. No. 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

Compositions according to the invention may comprise at least one builder. In the context of the present invention, no distinction will be made between builders and such components elsewhere called “co-builders”. Examples of builders are complexing agents, hereinafter also referred to as complexing agents, ion exchange compounds, and precipitating agents. Builders are selected from citrate, phosphates, silicates, carbonates, phosphonates, amino carboxylates and polycarboxylates.

In the context of the present invention, the term citrate includes the mono- and the dialkali metal salts and in particular the mono- and preferably the trisodium salt of citric acid, ammonium or substituted ammonium salts of citric acid as well as citric acid. Citrate can be used as the anhydrous compound or as the hydrate, for example as sodium citrate dihydrate. Quantities of citrate are calculated referring to anhydrous trisodium citrate.

The term phosphate includes sodium metaphosphate, sodium orthophosphate, sodium hydrogenphosphate, sodium pyrophosphate and polyphosphates such as sodium tripolyphosphate. Preferably, however, the composition according to the invention is free from phosphates and polyphosphates, with hydrogenphosphates being subsumed, for example free from trisodium phosphate, pentasodium tripolyphosphate and hexasodium metaphosphate (“phosphate-free”). In connection with phosphates and polyphosphates, “free from” should be understood within the context of the present invention as meaning that the content of phosphate and polyphosphate is in total in the range from 10 ppm to 0.2% by weight of the respective composition, determined by gravimetry.

The term carbonates includes alkali metal carbonates and alkali metal hydrogen carbonates, preferred are the sodium salts. Particularly preferred is Na₂CO₃.

Examples of phosphonates are hydroxyalkanephosphonates and aminoalkanephosphonates.

Among the hydroxyalkanephosphonates, the 1-hydroxyethane-1,1-diphosphonate (HEDP) is of particular importance as builder. It is preferably used as sodium salt, the disodium salt being neutral and the tetrasodium salt being alkaline (pH 9). Suitable aminoalkanephosphonates are preferably ethylene diaminetetramethylenephosphonate (EDTMP), diethylenetriaminepenta-methylenephosphonate (DTPMP), and also their higher homologues. They are preferably used in the form of the neutrally reacting sodium salts, e.g. as hexasodium salt of EDTMP or as hepta—and octa-sodium salts of DTPMP.

Examples of amino carboxylates and polycarboxylates are nitrilotriacetates, ethylene diamine tetraacetate, diethylene triamine pentaacetate, triethylene tetraamine hexaacetate, propylene diamines tetraacetic acid, ethanol-diglycines, methylglycine diacetate, and glutamine diacetate.

The term amino carboxylates and polycarboxylates also include their respective non-substituted or substituted ammonium salts and the alkali metal salts such as the sodium salts, in particular of the respective fully neutralized compound.

Silicates in the context of the present invention include in particular sodium disilicate and sodium metasilicate, alumosilicates such as for example zeolites and sheet silicates, in particular those of the formula α-Na₂Si₂O₅, β-Na₂Si₂O₅, and δ-Na₂Si₂O₅.

Compositions according to the invention may contain one or more builder selected from materials not being mentioned above. Examples of builders are α-hydroxypropionic acid and oxidized starch.

In one embodiment of the present invention, builder is selected from polycarboxylates. The term “polycarboxylates” includes non-polymeric polycarboxylates such as succinic acid, C2-C16-alkyl disuccinates, C2-C16-alkenyl disuccinates, ethylene diamine N,N′-disuccinic acid, tartaric acid diacetate, alkali metal malonates, tartaric acid monoacetate, propanetricarboxylic acid, butanetetracarboxylic acid and cyclopentanetetracarboxylic acid.

Oligomeric or polymeric polycarboxylates are for example polyaspartic acid or in particular alkali metal salts of (meth)acrylic acid homopolymers or (meth)acrylic acid copolymers.

Suitable co-monomers are monoethylenically unsaturated dicarboxylic acids such as maleic acid, fumaric acid, maleic anhydride, itaconic acid and citraconic acid. A suitable polymer is in particular polyacrylic acid, which preferably has a weight-average molecular weight Mw in the range from 2,000 to 40,000 g/mol, preferably 2,000 to 10,000 g/mol, in particular 3,000 to 8,000 g/mol. Further suitable copolymeric polycarboxylates are in particular those of acrylic acid with methacrylic acid and of acrylic acid or methacrylic acid with maleic acid and/or fumaric acid.

It is also possible to use copolymers of at least one monomer from the group consisting of monoethylenically unsaturated C3-C10-mono- or C4-C10-dicarboxylic acids or anhydrides thereof, such as maleic acid, maleic anhydride, acrylic acid, methacrylic acid, fumaric acid, itaconic acid and citraconic acid, with at least one hydrophilically or hydrophobically modified co-monomer as listed below.

Suitable hydrophobic co-monomers are, for example, isobutene, diisobutene, butene, pentene, hexene and styrene, olefins with ten or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C22-α-olefin, a mixture of C20-C24-α-olefins and polyisobutene having on average 12 to 100 carbon atoms per molecule.

Suitable hydrophilic co-monomers are monomers with sulfonate or phosphonate groups, and also non-ionic monomers with hydroxyl function or alkylene oxide groups. By way of example, mention may be made of: allyl alcohol, isoprenol, methoxypolyethylene glycol (meth)acrylate, methoxypolypropylene glycol (meth)acrylate, methoxypolybutylene glycol (meth)acrylate, methoxypoly(propylene oxide-co-ethylene oxide)(meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide)(meth)acrylate. Polyalkylene glycols here can comprise 3 to 50, in particular 5 to 40 and especially 10 to 30 alkylene oxide units per molecule.

Particularly preferred sulfonic-acid-group-containing monomers here are 1-acrylamido-1-propanesulfonic acid, 2-acrylamido-2-propanesulfonic acid, 2-acrylamido-2-methylpropanesulfonic acid, 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy)propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as sodium, potassium or ammonium salts thereof.

Particularly preferred phosphonate-group-containing monomers are vinylphosphonic acid and its salts.

Moreover, amphoteric polymers can also be used as builders.

Compositions according to the invention can comprise, for example, in the range from in total 0.1 to 70% by weight, preferably 10 to 50% by weight, preferably up to 20% by weight, of builder(s), especially in the case of solid formulations. Liquid formulations according to the invention preferably comprise in the range of from 0.1 to 8% by weight of builder.

Formulations according to the invention can comprise one or more alkali carriers. Alkali carriers ensure, for example, a pH of at least 9 if an alkaline pH is desired. Of suitability are, for example, the alkali metal carbonates, the alkali metal hydrogen carbonates, and alkali metal metasilicates mentioned above, and, additionally, alkali metal hydroxides. A preferred alkali metal is in each case potassium, particular preference being given to sodium. In one embodiment of the present invention, a pH>7 is adjusted by using amines, preferably alkanolamines, more preferably triethanolamine.

In one embodiment of the present invention, the laundry formulation according to the invention comprises additionally at least one enzyme.

Useful enzymes are, for example, one or more hydrolases selected from lipases, amylases, proteases, cellulases, hemicellulases, phospholipases, esterases, pectinases, lactases and peroxidases, and combinations of at least two of the foregoing types.

Such enzyme(s) can be incorporated at levels sufficient to provide an effective amount for cleaning. The preferred amount is in the range from 0.001% to 5% of active enzyme by weight in the detergent composition according to the invention. Together with enzymes also enzyme stabilizing systems may be used such as for example calcium ions, boric acid, boronic acid, propylene glycol and short chain carboxylic acids. In the context of the present invention, short chain carboxylic acids are selected from monocarboxylic acids with 1 to 3 carbon atoms per molecule and from dicarboxylic acids with 2 to 6 carbon atoms per molecule. Preferred examples are formic acid, acetic acid, propionic acid, oxalic acid, succinic acid, HOOC(CH₂)₃COOH, adipic acid and mixtures from at least two of the foregoing, as well as the respective sodium and potassium salts.

Compositions according to the invention may comprise one or more bleaching agent (bleaches).

Preferred bleaches are selected from sodium perborate, anhydrous or, for example, as the monohydrate or as the tetrahydrate or so-called dihydrate, sodium percarbonate, anhydrous or, for example, as the monohydrate, and sodium persulfate, where the term “persulfate” in each case includes the salt of the peracid H₂SO₅ and also the peroxodisulfate.

In this connection, the alkali metal salts can in each case also be alkali metal hydrogen carbonate, alkali metal hydrogen perborate and alkali metal hydrogen persulfate. However, the dialkali metal salts are preferred in each case.

Formulations according to the invention can comprise one or more bleach catalysts. Bleach catalysts can be selected from oxaziridinium-based bleach catalysts, bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and also cobalt-, iron-, copper- and ruthenium-amine complexes can also be used as bleach catalysts.

Formulations according to the invention can comprise one or more bleach activators, for example tetraacetyl ethylene diamine, tetraacetylmethylene diamine, tetraacetylglycoluril, tetraacetylhexylene diamine, acylated phenolsulfonates such as for example n-nonanoyl- or isononanoyloxybenzene sulfonates, N-methylmorpholinium-acetonitrile salts (“MMA salts”), trimethylammonium acetonitrile salts, N-acylimides such as, for example, N-nonanoylsuccinimide, 1,5-diacetyl-2,2-dioxohexahydro-1,3,5-triazine (“DADHT”) or nitrile quats (trimethylammonium acetonitrile salts).

Formulations according to the invention can comprise one or more corrosion inhibitors. In the present case, this is to be understood as including those compounds which inhibit the corrosion of metal. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxyhydroquinone, gallic acid, phloroglucinol or pyrogallol.

In one embodiment of the present invention, formulations according to the invention comprise in total in the range from 0.1 to 1.5% by weight of corrosion inhibitor.

Formulations according to the invention may also comprise further cleaning polymers and/or soil release polymers.

The additional cleaning polymers may include, without limitation, “multifunctional polyethylene imines” (for example BASF's Sokalan® HP20) and/or “multifunctional diamines” (for example BASF's Sokalan® HP96). Such multifunctional polyethylene imines are typically ethoxylated polyethylene imines with a weight-average molecular weight Mw in the range from 3,000 to 250,000, preferably 5,000 to 200,000, more preferably 8,000 to 100,000, more preferably 8,000 to 50,000, more preferably 10,000 to 30,000, and most preferably 10,000 to 20,000 g/mol.

Suitable multifunctional polyethylene imines have 80 wt.-% to 99 wt.-%, preferably 85 wt.-% to 45 99 wt.-%, more preferably 90 wt.-% to 98 wt.-%, most preferably 93 wt.-% to 97 wt.-% or 94 wt.-% to 96 wt.-% ethylene oxide side chains, based on the total weight of the materials. Ethoxylated polyethylene imines are typically based on a polyethylene imine core and a polyethylene oxide shell. Suitable polyethylene imine core molecules are polyethylene imines with a weight-average molecular weight Mw in the range of 500 to 5,000 g/mol. Preferably employed is a molecular weight from 500 to 1,000 g/mol, even more preferred is a Mw of 600 to 800 g/mol. The ethoxylated polymer then has on average 5 to 50, preferably 10 to 35 and even more preferably 20 to 35 ethylene oxide (EO) units per NH-functional group.

Suitable multifunctional diamines are typically ethoxylated C2 to C12 alkylene diamines, preferably hexamethylene diamine, which are further quatemized and optionally sulfated. Typical multifunctional diamines have a weight-average molecular weight Mw in the range from 2,000 to 10,000, more preferably 3,000 to 8,000, and most preferably 4,000 to 6,000 g/mol. In a preferred embodiment of the invention, ethoxylated hexamethylene diamine, furthermore quatemized and sulfated, may be employed, which contains on average 10 to 50, preferably 15 to 40 and even more preferably 20 to 30 ethylene oxide (EO) groups per NH-functional group, and which preferably bears two cationic ammonium groups and two anionic sulfate groups.

In a preferred embodiment of the present invention, the cleaning compositions may contain at least one multifunctional polyethylene imine and/or at least one multifunctional diamine to improve the cleaning performance, such as preferably improve the stain removal ability, especially the primary detergency of particulate stains on polyester fabrics of laundry detergents.

The multifunctional polyethylene imines or multifunctional diamines or mixtures thereof according to the descriptions above may be added to the laundry detergents and cleaning compositions in amounts of generally from 0.05 to 15 wt.-%, preferably from 0.1 to 10 wt.-% and more preferably from 0.25 to 5 wt.-% and even as low as up to 2 wt. %, based on the particular overall composition, including other components and water and/or solvents.

Thus, one aspect of the present invention is a laundry detergent composition, in particular a liquid laundry detergent, comprising (i) at least one inventive polymer and (ii) at least one compound selected from multifunctional polyethylene imines and multifunctional diamines and mixtures thereof.

In one embodiment of the present invention, the ratio of the at least one inventive polymer and (ii) the at least one compound selected from multifunctional polyethylene imines and multifunctional diamines and mixtures thereof, is from 10:1 to 1:10, preferably from 5:1 to 1:5 and more preferably from 3:1 to 1:3.

Laundry formulations comprising the inventive polymer may also comprise at least one antimicrobial agent (also often named preservatives).

The composition may contain one or more antimicrobial agents and/or preservatives as listed in patent WO2021/115912 A1 on pages 35 to 39.

Especially of interest are the following antimicrobial agents and/or preservatives: 4,4′-dichloro 2-hydroxydiphenyl ether (CAS-No. 3380-30-1), further names: 5-chloro-2-(4-chlorophenoxy) phenol, Diclosan, DCPP, which is commercially avail-able as a solution of 30 wt % of 4,4′-dichloro 2-hydroxydiphenyl ether in 1,2 propyl-eneglycol under the trade name Tinosan® HP 100 (BASF); 2-Phenoxyethanol (CAS-no. 122-99-6, further names: Phenoxyethanol, Methylphenylglycol, Phenoxetol, ethylene glycol phenyl ether, Ethylene glycol monophenyl ether, Protectol® PE); 2-bromo-2-nitropropane-1,3-diol (CAS-No. 52-51-7, further names: 2-bromo-2-nitro-1,3-propanediol, Bronopol®, Protectol® BN, Myacide AS); Glutaraldehyde (CAS-No. 111-30-8, further names: 1-5-pentandial, pentane-1,5-dial, glutaral, glutardialdehyde, Protectol® GA, Protectol® GA 50, Myacide® GA); Glyoxal (CAS No. 107-22-2; further names: ethandial, oxylaldehyde, 1,2-ethandial, Protectol® GL); 2-butyl-benzo[d]isothiazol-3-one (BBIT, CAS No. 4299-07-4); 2-methyl-2H-isothiazol-3-one (MIT, CAS No 2682-20-4); 2-octyl-2H-isothiazol-3-one (OIT, CAS No. 26530-20-1); 5-Chloro-2-methyl-2H-isothiazol-3-one (CIT, CMIT, CAS No. 26172-55-4); Mixture of 5-chloro-2-methyl-2H-isothiazol-3-one (CMIT, EINECS 247-500-7) and 2-methyl-2H-isothiazol-3-one (MIT, EINECS 220-239-6) (Mixture of CMIT/MIT, CAS No. 55965-84-9); 1,2-benzisothiazol-3(2H)-one (BIT, CAS No. 2634-33-5); Hexa-2,4-dienoic acid (Sorbic acid, CAS No. 110-44-1) and its salts, e.g. calcium sorbate, sodium sorbate, potassium (E,E)-hexa-2,4-dienoate (Potassium Sorbate, CAS No. 24634-61-5); Lactic acid and its salts; L-(+)-lactic acid (CAS No. 79-33-4); Benzoic acid and its sodium salt (CAS No 65-85-0, CAS No. 532-32-1) and salts of benzoic acid e.g. ammonium benzoate, calcium benzoate, magnesium benzoate, MEA-benzoate, potassium benzoate; Salicylic acid and its salts, e.g. calcium salicylate, magnesium salicylate, MEA sa-licylate, sodium salicylate, potassium salicylate, TEA salicylate; Benzalkonium chloride, bromide and saccharinate, e.g. benzalkonium chloride, benzalkonium bromide, benzalkonium saccharinate (CAS Nos 8001-54-5, 63449-41-2, 91080-29-4, 68989-01-5, 68424-85-1, 68391-01-5, 61789-y71-7, 85409-22-9); Didecyldimethylammonium chloride (DDAC, CAS No. 68424-95-3 and CAS No. 7173-51-5); N-(3-aminopropyl)-N-dodecylpropane-1,3-diamine (Diamine, CAS No. 2372-82-9); Peracetic acid (CAS No. 79-21-0); Hydrogen peroxide (CAS No. 7722-84-1).

The antimicrobial agent is added to the composition in a concentration of 0.001 to 10% relative to the total weight of the composition.

Preferably, the composition contains 2-Phenoxyethanol in a concentration of 0.1 to 2% or 4,4′-dichloro 2-hydroxydiphenyl ether (DCPP) in a concentration of 0.005 to 0.6%.

The invention thus further encompasses a method of preserving an aqueous composition according to the invention against microbial contamination or growth, which method comprises addition of 2-Phenoxyethanol. The invention thus further encompasses a method of providing an antimicrobial effect on textiles after treatment with a solid laundry detergent e.g. powders, granulates, capsules, tablets, bars etc.), a liquid laundry detergent, a softener or an after rinse containing 4,4′-dichloro 2-hydroxydiphenyl ether (DCPP).

Formulations according to the invention may also comprise water and/or additional organic solvents, e.g., ethanol or propylene glycol.

Further optional ingredients may be but are not limited to viscosity modifiers, cationic surfactants, foam boosting or foam reducing agents, perfumes, dyes, optical brighteners, and dye transfer inhibiting agents.

### (b) Dish Wash Compositions

Another aspect of the present invention is also a dish wash composition, comprising at least one polymer (as defined in any of the embodiments herein, especially the Embodiments 1 to 18; polymer in this section also named “inventive polymer”) as described above.

Thus, an aspect of the present invention is also the use of the inventive polymer as described above, in dish wash applications, such as manual or automatic dish wash applications.

Dish wash compositions according to the invention can be in the form of a liquid, semi-liquid, cream, lotion, gel, or solid composition, solid embodiments encompassing, for example, powders and tablets. Liquid compositions are typically preferred for manual dish wash applications, whereas solid formulations and pouch formulations (where the pouches may contain also solids in addition to liquid ingredients) are typically preferred for automatic dish washing compositions; however, in some areas of the world also liquid automatic dish wash compositions are used and are thus of course also encompassed by the term “dish wash composition”.

The dish wash compositions are intended for direct or indirect application onto dishware and metal and glass surfaces, such as drinking and other glasses, beakers, dish and cooking ware like pots and pans, and cutlery such as forks, spoons, knives and the like.

The inventive method of cleaning dishware, metal and/or glass surfaces comprises the step of applying the dish wash cleaning composition, preferably in liquid form, onto the surface, either directly or by means of a cleaning implement, i.e., in neat form. The composition is applied directly onto the surface to be treated and/or onto a cleaning device or implement such as a dish cloth, a sponge or a dish brush and the like without undergoing major dilution (immediately) prior to the application. The cleaning device or implement is preferably wet before or after the composition is delivered to it. In the method of the invention, the composition can also be applied in diluted form.

Both neat and dilute application give rise to superior cleaning performance, i.e., the formulations of the invention containing at least one inventive polymer exhibit excellent degreasing properties.

The effort of removing fat and/or oily soils from the dishware, metal and/or glass surfaces is decreased due to the presence of the inventive polymer, even when the level of surfactant used is lower than in conventional compositions.

Preferably the composition is formulated to provide superior grease cleaning (degreasing) properties, long-lasting suds and/or improved viscosity control at decreased temperature exposures; preferably at least two, more preferably all three properties are present in the inventive dish wash composition. Optional—preferably present—further benefits of the inventive manual dish wash composition include soil removal, shine, and/or hand care; more preferably at least two and most preferably all three further benefits are present in the inventive dish wash composition.

In a preferred embodiment of the present invention, the inventive polymer is one component of an automatic dish wash formulation, preferably of a solid automatic dish wash formulation. The formulations comprising the inventive polymer exhibit significantly reduced glass corrosion.

In another preferred embodiment of the present invention, the inventive polymer is one component of a manual dish wash formulation that additionally comprises at least one surfactant, preferably at least one anionic surfactant.

In another embodiment of the present invention, the inventive polymer is one component of a manual dish wash formulation that additionally comprises at least one anionic surfactant and at least one other surfactant, preferably selected from amphoteric surfactants and/or zwitterionic surfactants. In a preferred embodiment of the present invention, the manual dish wash formulations contain at least one amphoteric surfactant, preferably an amine oxide, or at least one zwitterionic surfactant, preferably a betaine, or mixtures thereof, to aid in the foaming, detergency, and/or mildness of the detergent composition.

Examples of suitable anionic surfactants are already mentioned above for laundry compositions.

Preferred anionic surfactants for manual dish wash compositions are selected from C10-C15 linear alkylbenzene sulfonates, C10-C18 alkylether sulfates with 1-5 ethoxy units and C10-C18 alkyl sulfates.

Preferably, the manual dish wash detergent formulation of the present invention comprises from at least 1 wt.-% to 50 wt.-%, preferably in the range from greater than or equal to about 3 wt.-% to equal to or less than about 35 wt.-%, more preferably in the range from greater than or equal to 5 wt.-% to less than or equal to 30 wt.-%, and most preferably in the range from greater than or equal to 5 wt.-% to less than or equal to 20 wt.-% of one or more anionic surfactants as described above, based on the particular overall composition, including other components and water and/or solvents.

Dish wash compositions according to the invention, preferably manual dish wash compositions, may comprise at least one amphoteric surfactant.

Examples of suitable amphoteric surfactants for dish wash compositions are already mentioned above for laundry compositions.

Preferred amphoteric surfactants for dish wash compositions are selected from C8-C18 alkyl-dimethyl aminoxides and C8-C18 alkyl-di(hydroxyethyl)aminoxide.

The manual dish wash detergent composition of the invention preferably comprises from 1 wt.-% 15 to 15 wt.-%, preferably from 2 wt.-% to 12 wt.-%, more preferably from 3 wt.-% to 10 wt.-% of the composition of an amphoteric surfactant, preferably an amine oxide surfactant. Preferably the composition of the invention comprises a mixture of the anionic surfactants and alkyl dimethyl amine oxides in a weight ratio of less than about 10:1, more preferably less than about 8:1, more preferably from about 5:1 to about 2:1.

Addition of the amphoteric surfactant provides good foaming properties in the dish wash composition.

Dish wash compositions according to the invention, preferably manual dish wash compositions, may comprise at least one zwitterionic surfactant.

Examples of suitable zwitterionic surfactants for dish wash compositions are already mentioned above for laundry compositions.

Preferred zwitterionic surfactants for dish wash compositions are selected from betaine surfactants, more preferable from Cocoamidopropylbetaine surfactants.

In a preferred embodiment of the present invention, the zwitterionic surfactant is Cocamidopropylbetaine.

The manual dish wash detergent composition of the invention optionally comprises from 1 wt.-% to 15 wt.-%, preferably from 2 wt.-% to 12 wt.-%, more preferably from 3 wt.-% to 10 wt.-% of the composition of a zwitterionic surfactant, preferably a betaine surfactant.

Dish wash compositions according to the invention, preferably manual dish wash compositions, may comprise at least one cationic surfactant.

Examples of suitable cationic surfactants for dish wash compositions are already mentioned above for laundry compositions.

Cationic surfactants, when present in the composition, are present in an effective amount, more preferably from 0.1 wt.-% to 5 wt.-%, preferably 0.2 wt.-% to 2 wt.-% of the composition.

Dish wash compositions according to the invention, preferably both manual and automatic dish wash compositions, may comprise at least one non-ionic surfactant.

Examples of suitable non-ionic surfactants for dish wash compositions are already mentioned above for laundry compositions. The non-ionic surfactant generally improves the rinsing properties of automatic dish wash formulations.

Preferred non-ionic surfactants for automatic dish wash compositions are C8-C18 alkyl ethoxylates, and ether capped poly(oxyalkylated) alcohol surfactants, e.g., the ones that are discussed in U.S. Pat. No. 6,482,994 and WO 01/42408.

Examples of suitable non-ionic surfactants for dish wash compositions are already mentioned above for laundry compositions. The non-ionic surfactant generally improves the foam properties of manual dish wash formulations.

Preferred non-ionic surfactants for manual dish wash formulations are the condensation products of Guerbet alcohols with from 2 to 18 moles, preferably 2 to 15, more preferably 5-12 of ethylene oxide per mole of alcohol. Other preferred non-ionic surfactants for use herein include fatty alcohol polyglycol ethers, alkyl polyglucosides and fatty acid glucamides.

The manual hand dish detergent composition of the present invention may comprise from 0.1 wt.-% to 10 wt.-%, preferably from 0.3 wt.-% to 5 wt.-%, more preferably from 0.4 wt.-% to 2 wt.-% of the composition, of a linear or branched C10 alkoxylated non-ionic surfactant having an average degree of alkoxylation of from 2 to 6, preferably from 3 to 5. Preferably, the linear or branched C10 alkoxylated non-ionic surfactant is a branched C10 ethoxylated non-ionic surfactant having an average degree of ethoxylation of from 2 to 6, preferably of from 3 to 5.

Preferably, the composition comprises from 60 wt.-% to 100 wt.-%, preferably from 80 wt.-% to 100 wt.-%, more preferably 100 wt.-% of the total linear or branched C10 alkoxylated non-ionic surfactant of the branched C10 ethoxylated non-ionic surfactant. The linear or branched C10 alkoxylated non-ionic surfactant preferably is a 2-propylheptyl ethoxylated non-ionic surfactant having an average degree of ethoxylation of from 3 to 5. A suitable 2-propylheptyl ethoxylated non-ionic surfactant having an average degree of ethoxylation of 4 is Lutensol® XP40, commercially available from BASF SE, Ludwigshafen, Germany. The use of a 2-propylheptyl ethoxylated non-ionic surfactant having an average degree of ethoxylation of from 3 to 5 leads to improved foam levels and long-lasting suds.

Thus, one aspect of the present invention is a manual dish wash detergent composition, in particular a liquid manual dish wash detergent composition, comprising (i) at least one inventive polymer, and (ii) at least one further 2-propylheptyl ethoxylated non-ionic surfactant having an average degree of ethoxylation of from 3 to 5.

In one embodiment of the present invention, the inventive polymer is one component of a dish wash formulation, preferably of an automatic dish wash formulation, that additionally comprises at least one builder, preferably at least one chelating agent.

Examples of suitable builders are already mentioned above for laundry compositions.

Preferred builders for dish wash compositions are selected from citrate, phosphates, silicates, carbonates, phosphonates, amino carboxylates and polycarboxylates. Even more preferred are builders selected from citrate, silicates, carbonates, phosphonates, amino carboxylates and polycarboxylates (i.e., phosphate-free compositions).

Particularly preferred is Na₂CO₃ and sodium citrate. Among the hydroxyalkane phosphonates, the 1-hydroxyethane-1,1-diphosphonate (HEDP) is of particular importance as builder. Preferred amino carboxylates are methylglycine diacetate (MGDA) and glutamine diacetate (GLDA). The term amino carboxylates and polycarboxylates also include their respective non-substituted or substituted ammonium salts and the alkali metal salts such as the sodium salts, in particular of the respective fully neutralized compound. Preferred polycarboxylates are for example polyaspartic acid or in particular alkali metal salts of (meth)acrylic acid homopolymers or (meth)acrylic acid copolymers. A suitable polymer is in particular polyacrylic acid, which preferably has a weight-average molecular weight Mw in the range from 2,000 to 40,000 g/mol, preferably 2,000 to 10,000 g/mol, in particular 3,000 to 8,000 g/mol.

Silicates in the context of the present invention include in particular sodium disilicate and sodium metasilicate, alumosilicates such as for example zeolites and sheet silicates, in particular those of the formula α-Na₂Si₂O₅, β-Na₂Si₂O₅, and δ-Na₂Si₂O₅.

Compositions according to the invention can comprise, for example, in the range from in total 0.1 to 70% by weight, preferably 10 to 50% by weight, preferably up to 20% by weight, of builder(s), especially in the case of solid formulations. Liquid formulations according to the invention preferably comprise in the range of from 0.1 to 8% by weight of builder.

Dish wash compositions according to the invention, preferably automatic dish wash compositions, comprise one or more bleaching agents (bleaches).

Examples of suitable bleaching agents are already mentioned above for laundry compositions.

Preferred bleaching agent for dish wash compositions is sodium percarbonate, anhydrous or, for example, as the monohydrate.

In one embodiment of the present invention, the inventive polymer is one component of an automatic dish wash formulation that additionally comprises one or more bleach catalysts.

Examples of suitable bleach catalysts are already mentioned above for laundry compositions.

Dish wash compositions according to the invention, especially automatic dish wash compositions, comprise one or more bleach activators.

Examples of suitable bleach activators are already mentioned above for laundry compositions.

Preferred bleach activator for dish wash compositions is tetraacetyl ethylene diamine.

Dish wash formulations according to the invention may comprise one or more corrosion inhibitors. In the present case, this is to be understood as including those compounds which inhibit the corrosion of metal and/or glass. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxyhydroquinone, gallic acid, phloroglucinol, pyrogallol or zinc salts, especially zinc benzoate, zinc gluconate, zinc lactate, zinc formicate, ZnCl₂, ZnSO₄, zinc acetate, zinc citrate, Zn(NO₃)₂, Zn(CH₃SO₃)₂, zinc gallate, zinc oxide, zinc hydroxide and zinc carbonate.

In one embodiment of the present invention, the dish wash formulation according to the invention comprises additionally at least one enzyme.

Useful enzymes are, for example, one or more hydrolases selected from lipases, amylases, proteases, cellulases, hemicellulases, phospholipases, esterases, pectinases, lactases and peroxidases, and combinations of at least two of the foregoing types, preferably at least one protease and one amylase.

Such enzyme(s) can be incorporated at levels sufficient to provide an effective amount for cleaning. The preferred amount is in the range from 0.001% to 5% of active enzyme by weight in the detergent composition according to the invention. Together with enzymes also enzyme stabilizing systems may be used such as for example calcium ions, boric acid, boronic acid, propylene glycol and short chain carboxylic acids. In the context of the present invention, short chain carboxylic acids are selected from monocarboxylic acids with 1 to 3 carbon atoms per molecule and from dicarboxylic acids with 2 to 6 carbon atoms per molecule. Preferred examples are formic acid, acetic acid, propionic acid, oxalic acid, succinic acid, HOOC(CH₂)₃COOH, adipic acid and mixtures from at least two of the foregoing, as well as the respective sodium and potassium salts.

Dish wash formulations according to the invention may comprise one or more alkali carriers.

Alkali carriers ensure, for example, a pH of at least 9 if an alkaline pH is desired. Of suitability are, for example, the alkali metal carbonates, the alkali metal hydrogen carbonates, and alkali metal metasilicates mentioned above, and, additionally, alkali metal hydroxides. A preferred alkali metal is in each case potassium, particular preference being given to sodium. In one embodiment of the present invention, a pH>7 is adjusted by using amines, preferably alkanolamines, more preferably triethanolamine.

Dish wash compositions according to the invention may comprise at least one hydrotrope in an effective amount, to ensure the compatibility of the liquiddish wash detergent compositions with water.

Suitable hydrotropes for use herein include anionic hydrotropes, particularly sodium, potassium, and ammonium xylene sulfonate, sodium, potassium and ammonium toluene sulfonate, sodium, potassium, and ammonium cumene sulfonate, and mixtures thereof, and related compounds, as disclosed in U.S. Pat. No. 3,915,903.

The liquid dish wash detergent compositions of the present invention typically comprise from 0.1 wt.-% to 15 wt.-% of the total liquid detergent composition of a hydrotrope, or mixtures thereof, preferably from 1 wt.-% to 10 wt.-%, most preferably from 2 wt.-% to 5 wt.-% of the total liquid manual dish wash composition.

Dish wash compositions according to the invention may comprise at least one organic solvent.

Examples of organic solvents are C4-C14 ethers and diethers, glycols, alkoxylated glycols, C6-C16 glycol ethers, alkoxylated aromatic alcohols, aromatic alcohols, aliphatic branched alcohols, alkoxylated aliphatic branched alcohols, alkoxylated linear C₁-C₅ alcohols, linear C₁-C₅ alcohols, amines, C8-C14 alkyl and cycloalkyl hydrocarbons and halohydrocarbons, and mixtures thereof.

When present, the liquid dish wash compositions will contain from 0.01 wt.-% to 20 wt.-%, preferably from 0.5 wt.-% to 15 wt.-%, more preferably from 1 wt.-% to 10 wt.-%, most preferably from 1 wt.-% to 5 wt.-% of the liquid detergent composition of a solvent. These solvents may be used in conjunction with an aqueous liquid carrier, such as water, or they may be used without any aqueous liquid carrier being present. At higher solvent systems, the absolute values of the viscosity may drop but there is a local maximum point in the viscosity profile. The dish wash compositions herein may further comprise from 30 wt.-% to 90 wt.-% of an aqueous liquid carrier, comprising water, in which the other essential and optional ingredients are dissolved, dispersed or suspended. More preferably the compositions of the present invention comprise from 45 wt.-% to 85 wt.-%, even more preferably from 60 wt.-% to 80 wt.-% of the aqueous liquid carrier. The aqueous liquid carrier, however, may contain other materials which are liquid, or which dissolve in the liquid carrier, at room temperature (25° C.) and which may also serve some other function besides that of an inert filler.

Dish wash compositions according to the invention may comprise at least one electrolyte.

Suitable electrolytes are preferably selected from inorganic salts, even more preferably selected from monovalent salts, most preferably sodium chloride. The dish wash compositions according to the invention may comprise from 0.1 wt.-% to 5 wt.-%, preferably from 0.2 wt.-% to 2 wt.-% of the composition of an electrolyte.

Liquid dish wash formulations comprising the inventive polymer may also comprise at least one antimicrobial agent.

Examples of suitable antimicrobial agents for dish wash compositions are already mentioned above for laundry compositions.

The antimicrobial agent may be added to the inventive liquid dish wash composition in a concentration of 0.0001 wt.-% to 10 wt.-% relative to the total weight of composition. Preferably, the formulation contains 2-phenoxyethanol in a concentration of 0.01 wt.-% to 5 wt.-%, more preferably 0.1 wt.-% to 2 wt.-% and/or 4,4′-dichloro 2-hydroxydiphenyl ether in a concentration of 0.001 wt.-% to 1 wt.-%, more preferably 0.002 wt.-% to 0.6 wt.-% (in all cases relative to the total weight of the composition).

Further additional ingredients are such as but not limited to conditioning polymers, cleaning polymers, surface modifying polymers, soil flocculating polymers, rheology modifying polymers, enzymes, structurants, cyclic diamines, structurants, emollients, humectants, skin rejuvenating actives, carboxylic acids, scrubbing particles, perfumes, malodor control agents, pigments, dyes, opacifiers, beads, pearlescent particles, microcapsules, disintegrants, pH adjusters including NaOH and alkanolamines such as monoethanolamines and buffering means.

### (c) General Cleaning Compositions and Formulations

In a preferred embodiment the at least one polymer (as defined in any of the embodiments herein, especially the Embodiments 1 to 18; polymer in this section also named “inventive polymer”) is used in a laundry detergent.

Liquid laundry detergents according to the present invention are composed of:

| | |
|---|---|
| 0.05-20% | of at least one inventive polymer |
| 1-50% | of surfactants |
| 0.1-40% | of builders, cobuilders and/or chelating agents |
| 0.1-50% | other adjuncts |
| water | to add up 100%. |

Preferred liquid laundry detergents according to the present invention are composed of:

| | |
|---|---|
| 0.2-6% | of at least one inventive polymer |
| 5-40% | of anionic surfactants selected from C10-C15- LAS and |
| | C10-C18 alkyl ethersulfates containing 1-5 ethoxy-units |
| 1.5-10% | of nonioic surfactants selected from C10-C18-alkyl |
| | ethoxylates containing 3-10 ethoxy-units |
| 2-20% | of soluble organic builders/cobuilders selected from C10-C18 |
| | fatty acids, di- and tricarboxylic acids, hydroxy-di- and |
| | hydroxytricaboxylic acids and polycarboxylic acids |
| 0.05-5% | of an enzyme system containing at least one enzyme suitable |
| | for detergent use and preferably also an enzyme stabilizing |
| | system |
| 0.5-20% | of mono- or diols selected from ethanol, isopropanol, |
| | ethylenglycol, or propylenglycol |
| 0.1-20% | other adjuncts |
| water | to add up to 100%. |

Solid laundry detergents (like e.g. powders, granules or tablets) according to the present invention are composed of:

| | |
|---|---|
| 0.05-20% | of at least one inventive polymer |
| 1-50% | of surfactants |
| 0.1-80% | of builders, cobuilders and/or chelating agents |
| 0-50% | fillers |
| 0-40% | bleach actives |
| 0.1-30% | other adjuncts and/or water |

wherein the sum of the ingredients adds up 100%.

Preferred solid laundry detergents according to the present invention are composed of:

| | |
|---|---|
| 0.2-6% | of at least one inventive polymer |
| 5-30% | of anionic surfactants selected from C10-C15- LAS, C10-C18 |
| | alkylsulfates and C10-C18 alkyl ethersulfates containing 1-5 |
| | ethoxy-units |
| 1.5-7.5% | of non-ionic surfactants selected from C10-C18-alkyl |
| | ethoxylates containing 3-10 ethoxy-units |
| 5-50% | of inorganic builders selected from sodium carbonate, |
| | sodiumbicarbonate, zeolites, soluble silicates, sodium sulfate |
| 0.5-15% | of cobuilders selected from C10-C18 fatty acids, di- and |
| | tricarboxylic acids, hydroxydi- and hydroxytricarboxylic acids |
| | and polycarboxylic acids |
| 0.1-5% | of an enzyme system containing at least one enzyme suitable |
| | for detergent use and preferably also an enzyme stabilizing |
| | system |
| 0.5-20% | of mono- or diols selected from ethanol, isopropanol, |
| | ethylenglycol, or propylenglycol |
| 0.1-20% | other adjuncts |
| water | to add up to 100% |

In a preferred embodiment the polymer according to the present invention is used in a manual dish wash detergent.

Liquid manual dish wash detergents according to the present invention are composed of:

| | |
|---|---|
| 0.05-10% | of at least one inventive polymer |
| 1-50% | of surfactants |
| 0.1-50% | of other adjuncts |
| water | to add up 100%. |

Preferred liquid manual dish wash detergents according to the present invention are composed of:

| | |
|---|---|
| 0.2-5% | of at least one inventive polymer |
| 5-40% | of anionic surfactants selected from C10-C15- LAS, C10-C18 alkyl ethersulfates |
| | containing 1-5 ethoxy-units, and C10-C18 alkylsulfate |
| 2-10% | of Cocamidopropylbetaine |
| 0-10% | of Lauramine oxide |
| 0-2% | of a non-ionic surfactant, preferably a C10-Guerbet alcohol alkoxylate |
| 0-5% | of an enzyme, preferably Amylase, and preferably also an enzyme stabilizing |
| | system |
| 0.5-20% | of mono- or diols selected from ethanol, isopropanol, ethylenglycol, or |
| | propylenglycol |
| 0.1-20% | other adjuncts |
| water | to add up to 100% |

The following table shows general cleaning compositions of certain types, which correspond to typical compositions correlating with typical washing conditions as typically employed in various regions and countries of the world. The at least one inventive polymer may be added to such formulation(s) in suitable amounts as outlined herein.

TABLE 1
General formula for laundry detergent compositions
according to the invention:

| | Ranges of Ingredient in Liquid |
|---|---|
| Ingredient | frame formulations |
| Linear alkyl benzene sulphonic acid | 0 to 30% |
| Coco fatty acid | 1 to 12% |
| Fatty alcohol ether sulphate | 0 to 25% |
| NaOH or mono or triethanol amine | Up to pH 7.5 to 9.0 |
| Alcohol ethoxylate | 3 to 10% |
| 1,2-Propylene glycol | 1 to 10% |
| Ethanol | 0 to 4% |
| Sodium citrate | 0 to 8% |
| water | Up to 100% |

TABLE 2
Liquid laundry frame formulations according to the invention:

| active | | | | | | |
|---|---|---|---|---|---|---|
| (numbers:% active) | F1 | F2 | F3 | F4 | F5 | F6 |
| alcohol ethoxylat 7EO | 5.40 | 10.80 | 12.40 | 7.30 | 1.60 | 7.60 |
| Coco fatty acid K12-18 | 2.40 | 3.10 | 3.20 | 3.20 | 3.50 | 6.40 |
| Fatty alcohol ether sulphate | 5.40 | 8.80 | 7.10 | 7.10 | 5.40 | 14.00 |
| Linear alkyl benzene | 5.50 | 0.00 | 14.50 | 15.50 | 10.70 | 0.00 |
| sulphonic acid | | | | | | |
| 1,2 Propandiol | 6.00 | 3.50 | 8.70 | 8.70 | 1.10 | 7.80 |
| Triethanolamine | | | | | | |
| Monoethanolamine | | | 4.00 | 4.30 | 0.30 | |
| NaOH | 2.20 | 1.10 | | | | 1.00 |
| Glycerol | | 0.80 | 3.00 | 2.80 | | |
| Ethanol | 2.00 | | | | 0.38 | 0.39 |
| Na citrate | 3.00 | 2.80 | 3.40 | 2.10 | 7.40 | 5.40 |
| Inventive Polymer (s) (total)* | 0-5 | 0-5 | 0-5 | 0-5 | 0-5 | 0-5 |
| Protease | 0-1 | 0-1 | 0-1 | 0-1 | 0-1 | 0-1 |
| Amylase | 0-0.5 | 0-0.5 | 0-0.5 | 0-0.5 | 0-0.5 | 0-0.5 |
| Cellulase | 0-0.3 | 0-0.3 | 0-0.3 | 0-0.3 | 0-0.3 | 0-0.3 |
| Lipase | 0-0.2 | 0-0.2 | 0-0.2 | 0-0.2 | 0-0.2 | 0-0.2 |
| Mannanase | 0-0.2 | 0-0.2 | 0-0.2 | 0-0.2 | 0-0.2 | 0-0.2 |
| Pectat Lyase | 0-0.3 | 0-0.3 | 0-0.3 | 0-0.3 | 0-0.3 | 0-0.3 |
| water | to 100 | to 100 | to 100 | to 100 | to 100 | to 100 |

| active | | | | | | | | |
|---|---|---|---|---|---|---|---|---|
| (numbers:% active) | F7 | F8 | F9 | F10 | F11 | F12 | F13 | F14 |
| alcohol ethoxylat 7EO | 3.80 | 0.30 | 13.30 | 8.00 | 5.70 | 20.00 | 9.20 | 29.00 |
| Coco fatty acid K12-18 | 2.80 | 3.00 | 1.70 | 1.80 | 2.50 | 5.00 | 8.60 | 10.40 |
| Fatty alcohol ether sulphate | 2.80 | 4.50 | 3.90 | 4.10 | | 10.00 | 22.20 | |
| Linear alkyl benzene | 6.30 | 5.43 | 11.45 | 5.90 | 10.10 | 10.00 | 28.00 | 27.00 |
| sulphonic acid | | | | | | | | |
| 1,2 Propandiol | 0.50 | | 2.50 | 0.40 | 6.00 | 10.00 | 7.00 | 7.00 |
| Triethanolamine | | | | 8.10 | | | | |
| Monoethanolamine | 0.40 | 1.80 | | | | | 8.00 | 7.00 |
| NaOH | | | 2.20 | | 3.30 | 1.50 | | |
| Glycerol | | 0.60 | 0.20 | 1.90 | | | 7.00 | 10.00 |
| Ethanol | | | 1.84 | | | | | |
| Na citrate | 4.60 | 3.30 | 3.30 | 1.40 | | 1.50 | | |
| Inventive Polymer (s) (total)* | 0-5 | 0-5 | 0-5 | 0-5 | 0-5 | 0-5 | 0-5 | 0-5 |
| Protease | 0-1 | 0-1 | 0-1 | 0-1 | 0-1 | 0-3 | 0-3 | 0-3 |
| Amylase | 0-0.5 | 0-0.5 | 0-0.5 | 0-0.5 | 0-0.5 | 0-0.5 | 0-0.5 | 0-0.5 |
| Cellulase | 0-0.3 | 0-0.3 | 0-0.3 | 0-0.3 | 0-0.3 | 0-0.3 | 0-0.3 | 0-0.3 |
| Lipase | 0-0.2 | 0-0.2 | 0-0.2 | 0-0.2 | 0-0.2 | 0-0.2 | 0-0.2 | 0-0.2 |
| Mannanase | 0-0.2 | 0-0.2 | 0-0.2 | 0-0.2 | 0-0.2 | 0-0.2 | 0-0.2 | 0-0.2 |
| Pectat Lyase | 0-0.3 | 0-0.3 | 0-0.3 | 0-0.3 | 0-0.3 | 0-0.3 | 0-0.3 | 0-0.3 |
| water | to 100 | to 100 | to 100 | to 100 | to 100 | to 100 | to 100 | to 100 |

*Without inventive polymer the formulations are comparative examples.

TABLE 3
Laundry powder frame formulations according to the invention:

| | Bleach free Powder | | | | | |
|---|---|---|---|---|---|---|
| Alcohol ethoxylate 7EO | 0.6 | 0 | 1 | 0 | 0 | 5.2 |
| Coco fatty acid K12-18 | 1.2 | 0 | 0 | 0 | 0 | 0 |
| Fatty alcohol ether sulphate | 1.5 | 0 | 0 | 0 | 0 | 6 |
| Linear alkyl benzene | 12.1 | 11.2 | 13.6 | 21.9 | 18.7 | 12.7 |
| sulphonic acid | | | | | | |
| Bleach activator | 0 | 0 | 0 | 0 | 0 | 0 |
| Percarbonate | 0 | 0 | 0 | 0 | 0 | 0 |
| AcetateNa | 0 | 0 | 0 | 0.1 | 0 | 0.1 |
| CitrateNa | 0 | 0 | 0 | 0 | 0 | 14 |
| Na Silicate | 27.9 | 5.8 | 6.6 | 2 | 15 | 20.3 |
| Na Carbonate | 17.2 | 35 | 37.3 | 30.1 | 37 | 1 |
| Na Phospahte | 0 | 0 | 0 | 14 | 0.3 | 0 |
| Na Hydrogencarbonate | 0.7 | 0.9 | 0.5 | 2.7 | 0.4 | 10.5 |
| Zeolite4A | 4.2 | 0.1 | 5.1 | 10.2 | 1.8 | 11.6 |
| HEDP | 0 | 0 | 0 | 0 | 0 | 0.13 |
| MGDA | 0 | 1.1 | 0 | 0 | 0 | 0 |
| Cellulase | 0-0.5 | 0-0.5 | 0-0.5 | 0-0.5 | 0-0.5 | 0-0.5 |
| Lipase | 0-0.4 | 0-0.4 | 0-0.4 | 0-0.4 | 0-0.4 | 0-0.4 |
| Mannanase | 0-0.4 | 0-0.4 | 0-0.4 | 0-0.4 | 0-0.4 | 0-0.4 |
| Protease | 0-1.5 | 0-1.5 | 0-1.5 | 0-1.5 | 0-1.5 | 0-1.5 |
| Amylase | 0-0.5 | 0-0.5 | 0-0.5 | 0-0.5 | 0-0.5 | 0-0.5 |
| Na Sulfate | 30.8 | 1.3 | 33 | 11 | 22 | 3 |
| Na Chloride | 0.2 | 43 | 0.1 | 0 | 0.1 | 0.1 |
| optical brightener | 0.02 | 0 | | 0.1 | 0.06 | |
| Inventive | 1 | 1 | 0.2 | 2 | 0.5 | 3 |
| Polymer(s) (total) | | | | | | |

| | Bleach containing Powder | | | | | |
|---|---|---|---|---|---|---|
| Alcohol ethoxylate 7EO | 1.2 | 5 | 4 | 0.5 | 0.5 | 0 |
| Coco fatty acid K12-18 | 0 | 0 | 0 | 0.3 | 0 | 0.6 |
| Fatty alcohol | 0 | 3.9 | 4.4 | 1.6 | 0 | 0 |
| ether sulphate | | | | | | |
| Linear alkyl benzene | 7.6 | 12.1 | 11.5 | 12.2 | 6.5 | 10.4 |
| sulphonic acid | | | | | | |
| Bleach activator | 0.2 | 9.5 | 9.5 | 0.5 | 0.8 | 2.2 |
| Percarbonate | 3.6 | 19.4 | 16.6 | 2.2 | 11.5 | 5.8 |
| AcetateNa | 0 | 6.7 | 7.1 | 0.3 | 1 | 0.7 |
| CitrateNa | 0 | 1.6 | 8.2 | 0.3 | 0.9 | 1.7 |
| Na Silicate | 3.6 | 11.3 | 16.4 | 10.2 | 9.1 | 16.5 |
| Na Carbonate | 21.6 | 8.7 | 1.4 | 8 | 22.9 | 14.8 |
| Na Phospahte | 0 | 0 | 0 | 0 | 0 | 0 |
| Na Hydrogencarbonate | 0.2 | 2.8 | 1.6 | 0.8 | 0.5 | 0.5 |
| Zeolite4A | 1.6 | 1.4 | 2.4 | 1.6 | 1.8 | 2.3 |
| HEDP | 0 | 0.27 | 0.16 | 0 | 0 | 0.17 |
| MGDA | 0 | 0 | 0 | 0 | 0 | 0 |
| Cellulase | 0-0.5 | 0-0.5 | 0-0.5 | 0-0.5 | 0-0.5 | 0-0.5 |
| Lipase | 0-0.4 | 0-0.4 | 0-0.4 | 0-0.4 | 0-0.4 | 0-0.4 |
| Mannanase | 0-0.4 | 0-0.4 | 0-0.4 | 0-0.4 | 0-0.4 | 0-0.4 |
| Protease | 0-1.5 | 0-1.5 | 0-1.5 | 0-1.5 | 0-1.5 | 0-1.5 |
| Amylase | 0-0.5 | 0-0.5 | 0-0.5 | 0-0.5 | 0-0.5 | 0-0.5 |
| Na Sulfate | 51 | 4 | 6 | 57 | 38 | 37 |
| Na Chloride | 1 | 1 | 0.5 | 1.2 | 0.2 | 1 |
| optical brightener | | 0.29 | 0.1 | 0.23 | 0.13 | 0.19 |
| Inventive | 2.2 | 9.2 | 2.2 | 0.7 | 1 | 0.4 |
| Polymer(s) (total) | | | | | | |

TABLE 4
Liquid manual dish wash frame formulations according to the invention:

| Ingredients | MDW.1 | MDW.2 | MDW.3 | MDW.4 | MDW.5 | MDW.6 | MDW.7 | MDW.8 |
|---|---|---|---|---|---|---|---|---|
| Linear C₁₂-C₁₄-alkyl- | 8 | 0 | 6 | 0 | 6 | 0 | 6 | 0 |
| benzenesulfonic acid | | | | | | | | |
| C₁₂-C₁₄-fatty alcohol × | 8 | 16 | 6 | 12 | 6 | 12 | 6 | 12 |
| 2 EO sulfate | | | | | | | | |
| Cocamidopropyl betaine | 0 | 0 | 4 | 4 | 0 | 0 | 2 | 2 |
| Lauramine oxide | 0 | 0 | 0 | 0 | 4 | 4 | 2 | 2 |
| 2-Propylheptanol × 4 EO | 0.5 | 0.5 | 0.5 | 0.5 | 0.5 | 0.5 | 0.5 | 0.5 |
| Inventive | 1 | 1 | 1 | 1 | 1 | 1 | 1 | 1 |
| Polymer (s) (total) | | | | | | | | |
| Ethanol | 2 | 2 | 2 | 2 | 2 | 2 | 2 | 2 |
| 2-Phenoxyethanol | 1 | 1 | 1 | 1 | 1 | 1 | 1 | 1 |
| (preservative) | | | | | | | | |
| Sodium chloride | 1 | 1 | 1 | 1 | 1 | 1 | 1 | 1 |
| Demin. water | add 100 | add 100 | add 100 | add 100 | add 100 | add 100 | add 100 | add 100 |
| Sodium hydroxide | add | add | add | add | add | add | add | add |
| | pH 8 | pH 8 | pH 8 | pH 8 | pH 8 | pH 8 | pH 8 | pH 8 |

The following examples shall further illustrate the present invention without restricting the scope of the invention.

### EXAMPLES

### Raw Materials

DI water

DI Water is deionized water with NH₃, with characterized by:

| | | |
|---|---|---|
| pH of | 9-10 | |
| electrical conductivity of | <15 μS/cm, as is | |
| electrical conductivity of | <0.5 μS/cm, | after removal of the conditioning chemical |
| | | ammonia (NH₃) with cation exchanger |
| Hardness (Ca + Mg) | max. 0.0005 mmol/l | |
| Sodium (Na) | max. 0.01 mg/l | |
| Ammonium (NH₄)* | max. 3 mg/l, | *conditioning chemical ammonia (NH₃) |
| Iron, total (Fe) | max. 1 mg/l | |
| Silicate, dissolved (SiO₂) | max. 0.05 mg/l | |
| Dissolved organic carbon (DOC) | max. 0.5 mg/l | |

### NaAA-DN95

NaAA-DN95 is an aqueous of sodium acrylate neutralization degree of 95% and a solid content of 38.2 wt. %. NaAA-DN95 are produced by neutralization of acrylic acid with 50 wt. %. an aqueous Solution of sodium hydroxide. The pH of NaAA-DN95 is in the range of 6.2-6.4

### Fe(II)SO₄

Fe(II)SO₄ is iron (II) sulfate heptahydrate with purity of: 99.0%

H₂O₂ (5%)

H₂O₂(5%) is an aqueous solution of hydrogen peroxide solution with an active concentration of 5 wt. %

### NaPS (7%)

NaPS (7%) is an aqueous solution of sodium persulfate solution with an active concentration of 7 wt. %

Catalytic additives (“organic compound”)
a) MGDA-Na₃ is an aqueous solution of trisodium dicarboxymethyl alaninate with solid content 40 wt. %
b) DTPA-Na₅ is an aqueous solution of penta sodium diethylenetriamine pentaacetate with solid content 50 wt. %
c) EDDS-Na₅ is an aqueous solution of ethylenediamine-N,N′-disuccinic acid trisodium with solid content 35 wt. %
d) CA-40% is an aqueous solution of citric acid solution with a solid content 40 wt. %
   Low molecular poly-α-qlucose

Low molecular poly-α-glucoses are poly-α-glucose with an average 2 to 30, preferrable 2 to 20 and mostly preferrable between 3-10 glycose units. It is produced from e.g. example from vegetable starch, for example from corn, wheat, tapioca and potatoes, and cellulose, by partial hydrolysis, e.g. by acid or enzymatic hydrolysis, and available as syrup or as dried powder

### Preferred are Corn Syrup and Corn Syrup Solids for Example:

1. GL01924 is Dry GL™ 01924E from the company Cargill, China Spray-dried glucose corn syrup obtained by enzymatic conversion of corn starch, with a Dextrose Equivalent (DE) of 25-30, a pH of 4.5-6.5, moisture content of ≤6 wt. %, with carbohydrate profile (dry basis) of 2 wt. % dextrose, 10 wt. % maltose, 13 wt. % maltotriose and 75 wt. % higher saccharides.
2. GL01925 is Dry GL™ 01925 from the company Cargill, USA Spray-dried glucose corn syrup obtained by enzymatic conversion of corn starch, with a Dextrose Equivalent (DE) of 23-27, a pH of 4.5-5.5, moisture content of ≤6 wt. %, with carbohydrate profile (dry basis) of 4.5 wt. % dextrose, 8.5 wt. % maltose and 87 wt. % higher saccharides.
3. MD240 is STAR-DRI 240 from the company Tate & Lyle, USA is a corn syrup powder with a Dextrose Equivalent (DE) of 22-30, a pH of 4.0-6.5 and moisture content of ≤6 wt. %, with carbohydrate profile (dry basis) of ≤5.5 wt. % dextrose, 9.5 wt. % maltose, 11 wt. % maltotriose and 74 wt. % higher saccharides.
4. C Dry™ GL 01921 from Cargill, France Spray-dried glucose corn syrup obtained by enzymatic conversion of corn starch, with a Dextrose Equivalent (DE) of 20-24, a pH of 3.5-5.5, moisture content of ≤6 wt. %, with carbohydrate profile (dry basis) of 2 wt. % dextrose, 7 wt. % maltose, 10 wt. % maltotriose and 81 wt. % higher saccharides.
5. C Dry™ GL 01924 from Cargill, France Spray-dried glucose corn syrup obtained by enzymatic conversion of corn starch, with a Dextrose Equivalent (DE) of 25.5-30.5, a pH of 3.5-5.5, moisture content of ≤6 wt. %, with carbohydrate profile (dry basis) of 3 wt. % dextrose, 11 wt. % maltose, 16.5 wt. % maltotriose and 69.5 wt. % higher saccharides.
6. C Dry™ GL 01932 from Cargill, France Spray-dried glucose corn syrup obtained by enzymatic conversion of corn starch, with a Dextrose Equivalent (DE) of 32-36, a pH of 3.5-5.5, moisture content of ≤6 wt. %, with carbohydrate profile (dry basis) of 1 wt. % dextrose, 25 wt. % maltose, 22 wt. % maltotriose and 52 wt. % higher saccharides
7. Cleardex™ 25-42 from Cargill, USA is an aqueous corn syrup solution, obtained by enzymatic conversion, with a Dextrose Equivalent (DE) of 23-27, a pH of 4.5-5.3, solid content of 77.4-78.6 wt. %, with carbohydrate profile (dry basis) of 6 wt. %, dextrose. 7 wt. % maltose, 9 wt. % maltotriose and 78 wt. % higher saccharides.
8. C* LMD 10982 from Cargill, Netherland is a liquid maltodextrin syrup solution from wheat obtained by enzymatic conversion, with a Dextrose Equivalent (DE) of 16.5-19.9, a pH of 3.5-5.5, solid content of 66.3-67.7 wt %

### Apparatus—Glass Reactor

2.5-liter glass reactor with 3-stages cross bean tutor
Magnetic diaphragm metering pump (gamma/L from ProMinent®, Germany) for dosing of NaAA-DN95
HPLC pump for dosing (PLM 707-1 from FLUSYS GmbH, Germany) for dosing of H₂O₂(5%) and NaPS (7%)
Refrigerated/heating circulators (Julabo FP 50)
   Apparatus—Steel reactor
6L-liter steel with 3-stages cross bean tutor
2 Magnetic diaphragm metering pump (gamma/4 from ProMinent® GmbH Germany) for dosing of NaPS (7%) and CA-40%
Refrigerated/heating circulators FP 50 from JULABO GmbH, Germany
2 HPLC pump (HPLC Compact pump 3350 from BISCHOFF Analysentechnik u.-geraete GmbH, Germany) for dosing for dosing of H₂O₂ (5%) and NaAA-DN95

### Methods

pH-Value

pH of the polymer solution with a solid content of 10 wt. % is measured in accordance with DIN 19268 (pH-measurement—pH-measurement of aqueous solutions with pH measuring chains with pH glass electrodes and evaluation of measurement uncertainty)

### Hazen Color and Gardner Color

Hazen Color and Gardner Color of the undiluted polymer solution measured in accordance with DIN EN 1557 at 23° C. (Surface active agents—Colorimetric characterization of optically clear coloured liquids (products) as X, Y, Z tristimulus values in transmission)

Concentration (dry)

The concentration of the polymer solution is measured with a Halogen Moisture Analyzer HR73 from Mettler-Toledo GmbH. 3.0 of the undiluted polymer is heated up to 120° C. for 15 min.

### K-Value (1%)

The K value of the polymer solution with a solid content of ≤5 wt. % is measured in accordance with DIN EN ISO 1628-1 (Plastics—Determination of the viscosity of polymers in dilute solution using capillary viscometers—Part 1)

Residual acrylate content:

The concentration of the residual acrylic acid resp. sodium acrylate during and after the polymerization is determined by HPLC measurement.

### Size Exclusion Chromatography (SEC)/Gel-Filtrations-Chromatographie

The number average molar mass Mₙ and mass average molar mass M. are given by the following equation:

$${M}_{n} = \frac{{\sum }_{i} {N}_{i} {M}_{i}}{{\sum }_{i} {N}_{i}} \\ and \\ {M}_{w} = \frac{{\sum }_{i} {N}_{i} {M}_{i}^{2}}{{\sum }_{i} {N}_{i} {M}_{i}}$$

where is the number of molecules of molecular mass

The dispersity D is given by the M_{w}Mₙ

The glucose units in average n is given by the following equation:

$$n = \frac{{M}_{n}}{{M}_{glucuose}} = \frac{{M}_{n}}{180 g / mol}$$

### Size Exclusion Chromatography (SEC)/Gel-Filtrations-Chromatographie (GPC) of Corn Syrup

For the determination of molecular weights, the hydrodynamic volume of a chain is set into relation with its molecular weight using narrowly distributed polymer standards with known molecular weights. The accuracy of the result for a sample depends on the similarity between the sample and the standard used for the calibration. The calibration was carried out using dextranes with molecular weights from M=180 g/mol to M=21,400 g/mol. The values outside this elution range were extrapolated.

The SEC/GPC measurements parameters:

| | |
|---|---|
| Eluent: | Dimethylacetamid + 1% LiCl |
| Detector: | DRI Agilent 1200 |
| Column-Temperature: | 35° C. |
| Flow rate: | 0.5 mL/min |
| Injection volume: | 100 μL |
| Concentration: | 4 mg/mL |
| Sample Filtration: | filtration over Sartorius Minisart RC 25 (0.2 μm) |

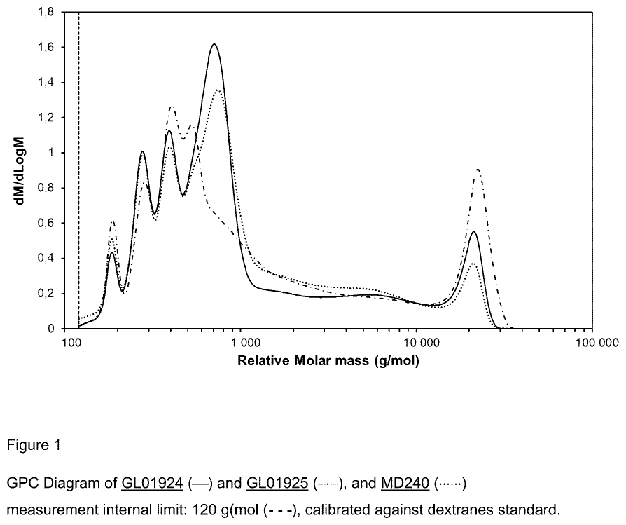

Following separating column combination (Plgel 3 μm) from Agilent Technologies, Inc, Santa Clara, CA, are used for the SEC/GPC measurements:
HPLC Guard Pre-Column, 8.0 mm ID×5 cm L
PIgel 3 μm, 7.5 mm ID×30 cm L
PIgel 3 μm, 7.5 mm ID×30 cm L
Plgel 3 μm, 7.5 mm ID×30 cm L FIG. 1 shows the GPC Diagram of GL01924, GL01925 and MD240

TABLE 5
GPC Data of GL01924, GL01925 and MD240

| | Mₙ | M_{w} | | |
|---|---|---|---|---|
| Example | (g/mol) | (g/mol) | D | n |
| GL01924 | 575 | 2650 | 4.6 | 3.2 |
| GL01925 | 579 | 3100 | 5.4 | 3.2 |
| SD240 | 615 | 4810 | 7.8 | 3.4 |
| Cleardex ™ 25-42 | 1200 | 2070 | 1.7 | 6.7 |

### Size Exclusion Chromatography (SEC)/Gel-Filtrations-Chromatographie (GPC) of Polymer

### Method A

For calibration of the SEC/GPC measurements of the invented polymer, pyridine with a molecular weight of M=79 g/mol and poly(2-vinylpyridine from PSS GmbH, Mainz, Germany is used as calibration standard in a range from M=620 g/mol to M=256,000 g/mol.

The SEC/GPC measurements parameters:

| | |
|---|---|
| Eluent: | 0.1% (w/w) Trifluoroacetic acid/ |
| | 0.1M NaCl in distillated water |
| Column-Temperature: | 35° C. |
| Flow rate: | 0.8 mL/min |
| Injection volume: | 100 μL |
| Concentration: | 1.5 mg/mL |
| Sample Filtration: | filtration over Sartorius Minisart RC 25 (0.2 μm) |

Following separating column combination from Tosoh Bioscience GmbH, Griesheim, Germany are used for the SEC/GPC measurements:
TSK G3000 PWxA, 7.8 mm ID×30 cm L
TSK G3000 PWxA, 7.8 mm ID×30 cm L
TSKgel G4000PWXL, 7.8 mm ID×30 cm L

### Method B

For calibration of the SEC/GPC measurements of the invented polymer, Polyethylene glycol (PEG) from PSS GmbH, Mainz, Germany is used as calibration standard in a range from M=106 g/mol to M=1.378.000 g/mol.

The SEC/GPC measurements parameters:

| | |
|---|---|
| Eluent: | 0.01 mol/l Phosphate-buffered saline, pH = 7.4 |
| | (=10 Na₂HPO₄ + 1.8 KH₂PO₄ + 2.7 KCl + 137 |
| | NaCl in mmol/L)/0.1M NaN₃ in distillated water |
| Column-Temperature: | 35° C. |
| Flow rate: | 0.5 mL/min |
| Injection volume: | 100 μL |
| Concentration: | 1.5 mg/mL |
| Sample Filtration: | filtration over Sartorius Minisart RC 25 (0.2 μm) |

Following separating column combination from Tosoh Bioscience GmbH, Griesheim, Germany are used for the SEC/GPC measurements:
TSK G3000 PWxA, 7.8 mm ID×30 cm L
20 TSK G3000 PWxA, 7.8 mm ID×30 cm L

### Example 1-6 (not inventive)

Preparation of water-soluble graft polymers without a Catalytic additives

### Example 1 (not inventive)

1100 g DI water is pre-charged in a 2.5-liter glass reactor. 596 g GL01924 and 0.018 g Fe(II)SO₄ are added under stirring (150 rpm). The reaction mixture is heated up to an internal temperature of 95° C. under N₂-atmosphere and stirring (150 ppm). After reaching the temperature 92° C. the parallel dosing of 498 g NaAA-DN95 for 240 min and 443 g H₂O₂(5%) for 255 min is started, followed by a post polymerization under stirring for 30 min. During the polymerization and post-polymerization, the reaction temperature keep constant in a range between 94-96° C. After cooling down in 30 min to room temperature. The product properties are summarized in Table 6

### Example 2 (not inventive)

The example is performed analogous to Example 1, except, that GL01925 is used instead of GL01924. The product properties are summarized in Table 6

### Example 3 (not inventive)

1100 g DI water is pre-charged in a 2.5-liter glass reactor. 596 g GL01925 and 0.018 g Fe(II)SO₄ are added under stirring (150 rpm). The reaction mixture is heated up to an internal temperature of 90° C. under N₂-atmosphere and stirring (150 ppm). After reaching the temperature 87° C. the parallel dosing of 498 g NaAA-DN95 for 240 min and 443 g H₂O₂(5%) for 255 min is started, followed by a post polymerization under stirring for 30 min. During the polymerization and post-polymerization, the reaction temperature keep constant in a range between 89-91° C. After cooling down in 30 min to room temperature. The product properties are summarized in Table 6

### Example 4 (not inventive)

The example is performed analogous to Example 2, except, that no Fe(II)SO₄ is added. The product properties are summarized in Table 6

### Example 5 (not inventive)

The example is performed analogous to Example 1, except, that SD240 is used instead of GL01924. The product properties are summarized in Table 6

### Example 6 (not inventive)

The example is performed analogous to Example 1, except the post polymerization step.

After end of the polymerization, the mixture is cooled down from 95° C. to 90° C. and the parallel adding of 159.0 g NaPS (7%) for 75 min and 79.5 g H₂O₂(5%) for 90 min is started.

After end of the H₂O₂ dosage, the reaction mixture is stirred for further 30 min at 90° C. and cooled down to RT in approx. 30 min. The product properties are summarized in Table 6

### Example 7 (not inventive)

1100 g DI water is pre-charged in a 2.5-liter glass reactor. 596 g GL01925, 6.619 g MGDA-Na₃ and 0,018 g Fe(II)SO₄ are added under stirring (150 rpm).

The reaction mixture is heated up to an internal temperature of 95° C. under N₂-atmosphere and stirring (150 ppm). After reaching the temperature 92° C. the parallel dosing of the NaAA-DN95 for 240 min and 443 g H₂O₂(5%) for 255 min is started.

After 3 h of dosing NaAA-DN95 and H₂O₂(5%), the reaction mixture became very discolored, and the polymerization is stopped after 3 h. Gardner color of the reaction mixture is >16.

### Example 8 (inventive)

1100 g DI water is pre-charged in a 2.5-liter glass reactor. 596 g GL01925, 0.655 g DTPA-Naₛ and 0.018 g Fe(II)SO₄ are added under stirring (150 rpm).

The Fe: catalytic additives mol ratio is 1:10. The reaction mixture is heated up to an internal temperature of 95° C. under N₂-atmosphere and stirring (150 ppm). After reaching the temperature 92° C. the parallel dosing of the NaAA-DN95 for 240 min and 443 g H₂O₂(5%) for 255 min is started. After end of the polymerization, the mixture is cooled down from 95° C. to 90° C. and the parallel adding of 159.0 g NaPS (7%) for 75 min and 79.5 g H₂O₂(5%) for 90 min is started. After end of the H₂O₂ dosage, the reaction mixture is stirred for further 30 min at 90° C. and cooled down to RT in approx. 30 min. The product properties are summarized in Table 7:

### Example 9 (inventive)

The example is performed analogous to Example 8, except 6.6 g of EDDS-Na₃ instead of DTPA-Nas. The Fe:catalytic additives mol ratio is 1:100. The product properties are summarized in Table 8:

### Example 10 (inventive)

1100 g DI water is pre-charged in a 2.5-liter glass reactor. 596 g GL01925, 3.42 g CA-40% and 0.018 g Fe(II)SO₄ are added under stirring (150 rpm). The Fe:catalytic additives mol ratio is 1:100. The reaction mixture is heated up to an internal temperature of 95° C. under N₂-atmosphere and stirring (150 ppm). After reaching the temperature 92° C. the parallel dosing of 498 g NaAA-DN95 for 240 min and 443 g H₂O₂(5%) for 255 min is started. After end of the polymerization, the mixture is cooled down from 95° C. to 90° C. and the parallel adding of 159.0 g NaPS (7%) for 75 min and 79.5 g H₂O₂(5%) for 90 min is started. The product properties are summarized in Table 8:

### Example 11 (inventive)

The example is performed analogous to Example 10, except 34.2 g of CA-40% is added before step post polymerization step started. The Fe:catalytic additives mol ratio is 1:100 during the polymerization and 1:1100 in the final product. The product properties are summarized in Table 8:

### Example 12 (inventive)

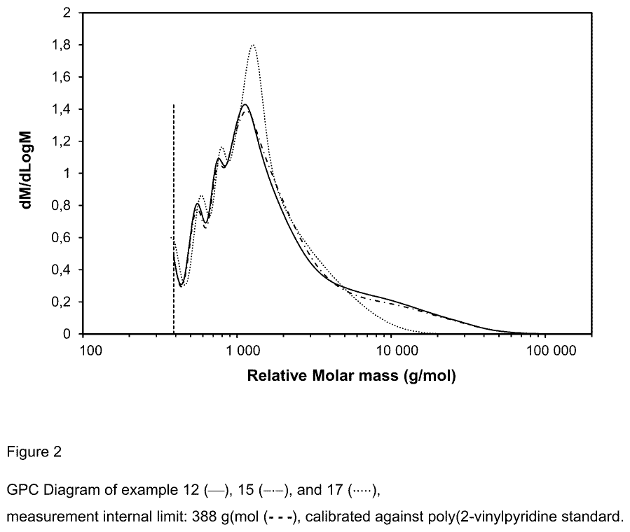

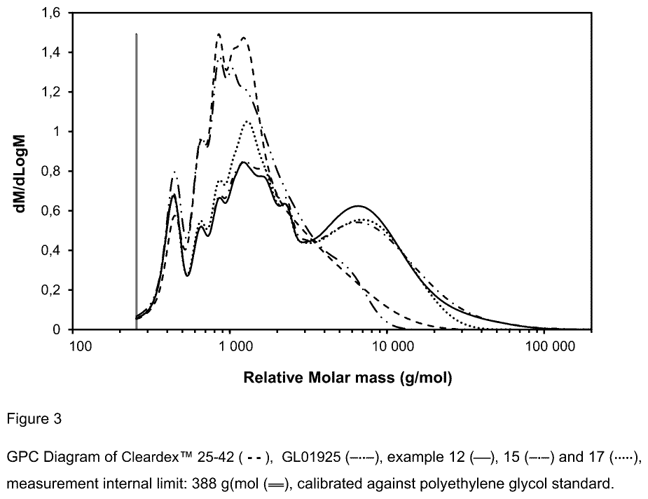

The example is performed analogous to Example 11, except the DI water content in the pre-charge is reduced from 1100 g to 492 g. The Fe:catalytic additives mol ratio is 1:100 during the polymerization and 1:1100 in the final product. The product properties are summarized in the product properties are summarized in Table 8: FIGS. 2 and 3 shows the GPC Diagram of example 12.

### Example 13 (inventive)

The example is performed analogous to Example 11, except, that SD240 is used instead of GL01925 and the DI water content in the pre-charge is reduced from 1100 g to 827 g. The product properties are summarized in Table 8:

### Example 14 (inventive)

1264 g DI water is pre-charged in a 6L steel vessel. 911 g GL01925, 5.2 g CA-40% and 0,028g Fe(II)SO₄ are added under stirring (150 rpm) for 1 h. The Fe:catalytic additives mol ratio is 1:100. The reactor is inerted with 2×2.5 bar nitrogen gas. After the second release, an overpressure of 0.3 bar is left. The closed reaction vessel is heated up to an internal temperature of 95° C. stirring (150 ppm). After reaching the temperature 95° C., the internal pressure increased to 2.4 bar and the parallel dosing of 762 g NaAA-DN95 for 240 min and 678 g H₂O₂(5%) for 255 min is started. After end of the polymerization, the internal pressure increased to 2.9 bar. 52.3 g CA-40% is added after the polymerization to the reaction mixture. The reaction mixture is cooled from 95° C. to 90° C. and the parallel adding of 243 g NaPS (7%) for 75 min and 121 g H₂O₂(5%) for 90 min is started. After end of the H₂O₂ dosage, the reaction mixture is stirred for further 30 min at 90° C. and cooled down to RT in approx. 30 min. The product properties are summarized in Table 8:

### EXAMPLE 15 (INVENTIVE)

790 g DI water is pre-charged in a 6L steel vessel. 1055 g GL01925, 6.0 g CA-40% and 0.032 g Fe(II)SO₄ are added under stirring (150 rpm) for 1 h. The Fe:catalytic additives mol ratio is 1:100. The reactor is inerted with 2×2.5 bar nitrogen. After the second release, an overpressure of 0.3 bar nitrogen is left. The closed reaction vessel is heated up to an internal temperature of 95° C. stirring (150 ppm). After reaching the temperature 95° C., the internal pressure increased to 2.4 bar and the parallel dosing of 883 g NaAA-DN95 for 240 min and 785 g H₂O₂(5%) for 255 min is started. After end of the polymerization, the internal pressure increased to 3.1 bar. 60.5 g CA-40% is added after the polymerization to the reaction mixture. The reaction mixture is cooled from 95° C. to 90° C. and the parallel adding of 281 g NaPS (7%) for 75 min and 140 g H₂O₂(5%) for 90 min is started. After end of the H₂O₂ dosage, the reaction mixture is stirred for further 30 min at 90° C. and cooled down to RT in approx. 30 min. The product properties are summarized in Table 8:

FIGS. 2 and 3 shows the GPC Diagram of example 15.

### EXAMPLE 16 (INVENTIVE)

669 g DI water is pre-charged in a 6L steel vessel. 1461 g Cleardex™ 25-42, 5,3 g citric acid anhydride and 0.036 g Fe(II)SO₄ are added under stirring (150 rpm) for 1 h. The Fe:catalytic additives mol ratio is 1:100. The reactor is inertized with 2×2.5 bar nitrogen. After the second release, an overpressure of 0.3 bar nitrogen is left. The closed reaction vessel is heated up to an internal temperature of 95° C. stirring (150 ppm). After reaching the temperature 95° C., the internal pressure increased to 2.4 bar and the parallel dosing of 1015 g NaAA-DN95 for 240 min and 449 g H₂O₂(10%) for 255 min is started. After end of the polymerization, the internal pressure increased to 3.1 bar. 63 g CA-40% is added after the polymerization to the reaction mixture. The reaction mixture is cooled from 95° C. to 90° C. and the parallel adding of 319 g NaPS (7%) for 75 min and 20 g H₂O₂(10%) for 90 min is started. After end of the H₂O₂ dosage, the reaction mixture is stirred for further 30 min at 90° C. and cooled down to RT in approx. 30 min. The product properties are summarized in Table 8:

### Example 17 (inventive)

995,4 g C* LMD 10982 is pre-charged in a 2.5-liter glass reactor. 6,4 g CA-40% and 0.021 g Fe(II)SO₄ are added under stirring (150 rpm). The reaction mixture is heated up to an internal temperature of 95° C. under N₂-atmosphere and stirring (150 ppm). After reaching the temperature 92° C. the parallel dosing of 564 g NaAA-DN95 for 240 min and 502 g H₂O₂(5%) for 255 min is started. After end of the polymerization, the mixture is cooled down from 95° C. to 90° C. and the adding of

180.0 g NaPS (7%) for 75 min is started. After end of the NaPS dosage, the reaction mixture is stirred for further 30 min at 90° C. and cooled down to RT in approx. 30 min. The trial was repeated 2 times and average product properties of these 3 batches are summarized in Table 8:

### Example 18 (Granulation of example 17, Inventive)

A lab scale granulator, commercially available as “WFP-Mini” from the company DMR, is charged with 300 g of solid Sokalan PA 15 Granules, that are milled down using a Kinetatica Polymix PX-MFL 90D at 4000 rpm (rounds per minute), 2 mm mesh. An amount of 26-27 Nm³/h of nitrogen with a temperature of 106-110° C. is blown from the bottom. A fluidized bed of Sokalan PA 15 particles is obtained. Then, a polymer solution of example 17 is introduced by spraying about 11g/minute liquid (at about 20° C. feed temperature) into the fluidized bed from the bottom through a three-fluid nozzle. The pressure of the atomizing gas is 1,5-2 bar.

Granules are formed, and the bed temperature, which corresponds to the surface temperature of the solids in the fluidized bed, is 75-80° C.

About every 25 minutes an aliquot of granules (180-220 g) is removed from the vessel and classified by sieving. Three fractions are obtained: coarse particles (diameter>1 mm), value fraction (diameter 0,35-1 mm) and fines (diameter<0.35 mm). These coarse particles, are milled down using a hammer mill (Kinetatica Polymix PX-MFL 90D) at 4000 rpm (rounds per minute), 2 mm mesh. The powder so obtained and the fines are returned into the fluidized bed. The value fraction, which is not milled down, left the process and is collected.

After 4 kg of sprayed liquid, a steady state is reached. Then, the value fraction is collected as inventive granules. The product properties are summarized in Table 9:

### Liquid Detergent Formulation Compatibility Test

For the liquid detergent formulation compatibility test 1 or 3 wt. % of the polymer (calculated as dry substance) are stirred in the liquid detergent formulation. The appearance or the liquid detergent formulation is visible classified a) after preparation (0 d) and after 7 (7 d) and 28 days (28 d) in 4 classes: C: clear/ST: slightly turbid/T: turbid/SP: separated

### PA1

PA1 is an aqueous solution of polyacrylic acid, sodium salt with a pH of 7 and a K-Value of approx. 15 and a weight average molecular weight of 1200 g/mol

### Following Polymers are Tested in Liquid Detergent Formulation (Formulation A):

PA1, sodium salt (molar mass 1200 g/mol), Example 1 and 3 (not invented examples) and 8-15 (invented examples)

| | |
|---|---|
| Aqueous solution of | |
| 19.6 wt. % | sodium laureth sulfate (2 EO), sodium salt |
| 6.7 wt. % | C12-C14 Fatty alcohol + 7 EO |
| 5.0 wt. % | Linear C10C13 |
| alkyl benzene sulfonic acid | |
| 2.7 wt. % | coconut fatty acid |
| 2.2 wt. % | sodium hydroxide |
| 4.1 wt. % | sodium salt of citric acid |
| 2.7 wt. % | propylene glycol |
| 0.8 wt. % | ethanol |
| 0.4 wt. % | glycerin |

Water to 100%

### Formulation B

Market/commercial premium liquid laundry detergent specially formulated to contain no antiredeposition/antigreying polymer. Test polymers are added to this detergent base at time of use.

The liquid detergent formulation stability tests are summarized in table 3.

### Washing Performance Test Based on the Formulation A:

Test method for antigreying with red clay fabric

The antigreying performance for the selected polymers was determined as follows: Several white test swatches are washed together with a combination of 0,75 g red clay fabric and 1,25 g SBL 2004 and 20 steel balls at 40° C. in the liquid detergent formulation A with the selected polymers. After the wash the test fabrics are rinsed and spin-dried. This wash cycle is repeated two times with new soiled fabrics and new wash liquor. After the third wash the test fabrics are rinsed, spin-dried and dried in the air.

The antigreying performance is determined by measuring the remission value of the white test fabrics before and after wash with the spectrophotometer from Fa. Datacolor (Elrepho 2000) at 460 nm. The higher the value, the better is the performance.

### Washing Conditions:

| | |
|---|---|
| Test equipment | Launder-Ometer ®, LP2 |
| | from SDL Atlas Inc., Rock Hill South Carolina, USA |
| Washing liquor | 250 ml |
| Washing time/ | 20 min at 40° C. |
| temperature | |
| Detergent Dosage | 1 g/L |
| Fabric/liquor ratio | 1:10 |
| Washing cycles | 3 |
| Water hardness | 2.5 mmol/l with Ca²⁺:Mg²⁺:HCO₃⁻ − 4:1:8 |
| Soiling with | 0.75 g SBL Red clay fabric/ |
| | 1.25 g SBL2004 or 2.5 g clay slurry |
| Sum test fabric | WFK 10 A, WFK 12 A, WFK 80 A, EMPA 221 |
| | WFK 20 A, WFK 30 A |
| | EMPA 406 |
| White test fabric | each 10 × 10 cm |

### Washing Performance Test Based on the Formulation B:

Test method for antiredeposition with Georgia red clay.

The antiredeposition performance for the selected polymers was determined as follows: Several white test swatches are washed together with 0.5 g Georgia red clay (solid particulate) at 30° C. in the presence of liquid detergent Formulation B with the selected test polymers (at 1 wt % active material). After the wash the test fabrics are rinsed and rung-dried by hand. This wash cycle is repeated three times (for a total of four) with fresh Georgia red clay and new wash liquor. After the fourth wash the test fabrics are rinsed, spin-dried in wringing machine and dried in a machine dryer for 30 minutes at medium heat setting.

The antiredeposition performance is determined by measuring L, a, b values of the the white test fabrics before (i) and after (f) wash using a Konica Minolta portable spectrophotometer.

The dE value is then calculated from the resultant L, a, b values as follows:

$$dE = \sqrt{{( {L}_{f} - {L}_{i} )}^{2} + {( {a}_{f} - {a}_{i} )}^{2} + {( {b}_{f} - {b}_{i} )}^{2}}$$

Lower the dE values correspond to better antiredeposition performance toward Georgia red clay. Results are given in the tables.

Antiredeposition washing conditions using Formulation B are

| | |
|---|---|
| Test equipment | Terg-O-Tometer |
| | from Testfabrics, Inc., West Pittston, |
| | Pennsylvania, USA |
| Washing liquor | 1,000 ml |
| Washing time/ | 20 min at 30° C. |
| temperature | |
| Rinsing Time/ | 3 min at 15° C. |
| Temperature | |
| Detergent Dosage | 1 g/L |
| Fabric/liquor ratio | 1:33 |
| Washing cycles | 4 |
| Water hardness | 1.5 mmol/l with Ca²⁺:Mg² − 2:1 |
| Soiling with | 0.5 g Georgia Red Clay |
| Sum test fabric | WFK 10 A, (6 pieces each, each 7.62 × 10.16 cm) |
| | WFK 12 A, (3 pieces each, each 11.43 × 11.43 cm) |
| | WFK 20 A, (6 pieces each, each 7.62 × 10.16 cm) |
| White test fabric | each 10 × 10 cm |

### Test Method for Accelerated Aging and Chelator Performance

Chelator-iron binding performance for the selected polymer systems was determined as follows:

Chelators (see Table yy) were added to a 20 g sample of graft copolymer, using Example 1, at various dosing levels (0.025, 0.05, 0.1 or 0.2 weight % based on sample). Hydrogen peroxide was then loaded to a target content (0.05, 0.1, 0.2 weight % based on sample). The pH of each system was adjusted to 6.0-6.5 to stay within intended range for the graft biopolymer. The resultant sample of aqueous polymer, chelator, and peroxide was stored at 90° C. and monitored for changes in Gardner color over 4 to 6 hours. An ideal chelator-iron-peroxide system would maintain low Gardner color (<4 units) for an extended period of time, i.e. longer times are higher performing. The reported values (Table yy) are the inflection times at which the sample began to generate color> Gardner 4.

The Following Soiling Material and Test Fabric are from Wfk Testgewebe GmbH, Bruggen, Deutschland:
SBL2004: Soil Ballast wfk, 100% cotton, approx. 8 g soil/swatch
WFK 10 A: 100% cotton fabric DIN 53919/ISO 2267
WFK 12 A: 100% cotton terry cloth, bleached with optical brightener
WFK 80 A: 100% cotton Knitwear, Ponte de Roma
WFK 20 A: polyester/cotton 65/35 fabric
WFK 30 A: 100% polyester fabric
   The Following Test Fabrics are from Swissatest Testmaterialien AG, St. Gallen, Switzerland
EMPA 221: Cotton fabric, cretonne, bleached, without optical brightener
EMPA 406: Polyamide 6.6 spun, type 200, plain weave, ISO 105-F03
   The Following Soiling Material and Test Fabric is from Center for Testmaterials (C.F.T.) BV, Vlaardingen, Netherland
SBL Red Clay: Soil Ballast Load red clay on Cotton

### Determination of “Ready Biodegradability” According the OECD 301F Guidelines

Biodegradation in wastewater is tested in triplicate using the OECD 301F manometric respirometry method. OECD 301F is an aerobic test that measures biodegradation of a sample by measuring the consumption of oxygen. To a measured volume of medium, 100 mg/L of example 14, which is the nominal sole source of carbon is added along with the inoculum (aerated sludge taken from Mannheim wastewater treatment plant). This is stirred in a closed flask at a constant temperature (25° C.) for 28 days. The consumption of oxygen is determined by measuring the change in pressure in the apparatus using an Oxi TopC. Evolved carbon dioxide is absorbed in a solution of sodium hydroxide. Nitrification inhibitors are added to the flask to prevent usage of oxygen due to nitrification. The amount of oxygen taken up by the microbial population during biodegradation of the test substance (corrected for uptake by blank inoculum, run in parallel) is expressed as a percentage of ThOD (Theoritical oxygen demand, which is measured by the elemental analysis of the compound). A positive control Glucose/Glucosamine is run along with the test samples for each cabinet.

### Calculations: Theoretical Oxygen Demand

Amount of oxygen required to oxidize a compound to its final oxidation products.

This is calculated using the elemental analysis data.

$$\% Biodegradation = \frac{Biological oxygen demand ( from the experiment ) \times 100}{Theoretical oxygen demand}$$

### Duration of Test: 28 Days

Source of sludge: Mannheim wastewater treatment plant
Concentration of sludge: 30 mg/L

### Validity: According to the OECD Guidelines the Test is Valid if:

1. The reference reaches 60% by 14 days.
2. The difference of the extremes of the test replicates by the end of the test is less than 20%.
3. Oxygen uptake of inoculum blank is 20-30 mg O₂/L and must not be greater than 60 mg O₂/L.
4. The pH measured at the end of the test is between 6.0-8.5.

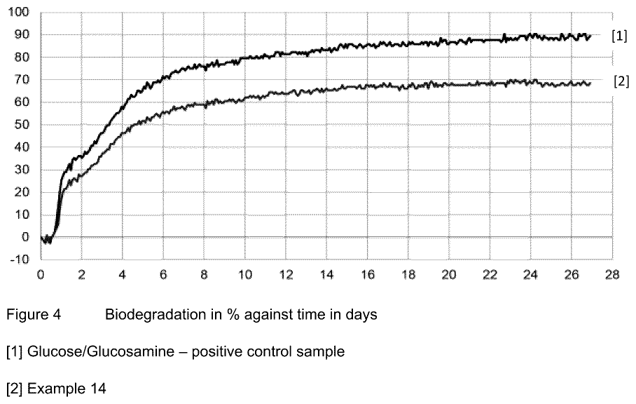

Example 12 showed 70% biodegradation and can be classified as “readily biodegradable” and fulfill the validity criterion detailed by OECD—see FIG. 4

TABLE 6
Product properties of example 1-6 (not inventive)

| Method | Performance | | | | | | |
|---|---|---|---|---|---|---|---|
| Example | 1 | 2 | 3 | 4 | 5 | 6 | Unit |
| Low molecular | GL01924 | GL01925 | GL01925 | GL01925 | SD240 | GL01924 | |
| poly-α-glucoses | | | | | | | |
| Polymerization | 95 | 95 | 90 | 95 | 95 | 95 | ° C. |
| Temperature | | | | | | | |
| Fe(II)SO₄:NaAA- | 1:82,000 | 1:82,000 | 1:82,000 | 1:82,000 | 1:82,000 | 1:82,000 | |
| DN95 - Mol ratio | | | | | | | |
| pH-Value as is | 6.6 | 6.2 | 6.4 | 6.1 | 6.4 | 6.4 | |

Color (1 day & 23° C.)

| | | | | | | |
|---|---|---|---|---|---|---|
| Hazen Color | 26 | — | 54 | — | — | |
| Gardner Color | 0.1 | 16.1 | 0.2 | 9.0 | 14.2 | >18 |

Color (180 day & 23° C.)

| | | | | | | | |
|---|---|---|---|---|---|---|---|
| Gardner Color | 8.2 | >18 | 8.7 | 15.1 | >18 | | |
| Concentration | 29.0 | 29.3 | 29.0 | 29.4 | 29.4 | 26.8 | % |
| (dry) | | | | | | | |
| K-Value (5%) | 12.3 | 11.5 | 13.5 | 11.6 | 12.4 | 12.0 | |
| Residual | 1.2 | 1.5 | 2.1 | 1.8 | 1.4 | 0.045 | % |
| acrylate content | | | | | | | |

TABLE 7
Product properties of example 8-15 (inventive)

| Method | Performance | | | | | | |
|---|---|---|---|---|---|---|---|
| Example | 8 | 9 | 10 | 11 | 12 | 13 | Unit |
| Low molecular | GL1925 | GL01925 | GL01925 | GL01925 | GL01925 | SD240 | |
| poly-α-glucoses | | | | | | | |
| Catalytic additives | DTPA-Na₅ | EDDS-Na₃ | CA-40% | CA-40% | CA-40% | CA-40% | |
| pH-Value as is | 6.4 | 6.5 | 6.2 | 5.9 | 5.7 | 6.1 | |

Color (1 day & 23° C.)

| | | | | | | |
|---|---|---|---|---|---|---|
| Hazen Color | 105 | 49 | 254 | 53 | 91 | 62 |
| Gardner Color | 0.5 | 0.3 | 1.3 | 0.2 | 0.3 | 0.2 |

Color (180 day & 23° C.)

| | | | | | | | |
|---|---|---|---|---|---|---|---|
| Gardner Color | 1.4 | 1.2 | 1.8 | 0.3 | 0.4 | 0.4 | |
| Concentration (dry) | 26.7 | 26.7 | 26.8 | 26.7 | 34.8 | 30.1 | % |
| K-Value (5%) | 12.1 | 12.5 | 12.0 | 11.8 | 11.9 | 11.4 | |
| Residual acrylate content | 170 | 490 | 310 | 250 | 100 | 210 | ppm |
| number average molar mass Mₙ | | | | | 1210*/1450** | | g/mol |
| mass average molar mass M_{w} | | | | | 3310*/5390** | | g/mol |
| D = M_{w}/Mₙ | | | | | 2.7*/3.8** | | |

The number average molar mass Mₙ and mass average molar mass M_{w} are given by the equations as disclosed before, measured with Method A* and Method B**

TABLE 8
Product properties of example 14-18 (inventive)

| Method | | | Performance | | | |
|---|---|---|---|---|---|---|
| Example | 14 | 15 | 16 | 17 | 18 | |
| Low molecular | GL01925 | GL01925 | Cleardex ™ 25-42 | C* LMD 10982 | | |
| poly-α-glucoses | | | | | | |
| Catalytic additives | CA-40% | CA-40% | Citric acid anhydride | CA-40% | | |
| pH-Value as is | 5.7 | 5.7 | 5.9 | 5.1 | | |
| | | | Color (1 day & 23° C.) | | | |
| Hazen Color | 32 | 245 | 154 | 68 | | |
| Gardner Color | 0.1 | 1.2 | 0.8 | 0.2 | | |
| | | | Color (180 day & 23° C.) | | | |
| Gardner Color | 0.2 | 1.6 | 0.9 | 0.3 | | |
| Concentration (dry) | 30.4 | 35.9 | 40.6 | 41.2 | 97.3 | % |
| K-Value (5%) | 11.7 | 11.5 | 11.8 | 11.3 | | |
| Residual acrylate content | 180 | 80 | <10 | 40 | | ppm |
| Residual peroxide | | | 777 | <1000 | | ppm |
| number average molar mass Mₙ | 1220*/1410** | 1220*/1390** | 1110*/1340** | | | g/mol |
| mass average molar mass M_{w} | 3310*/5300** | 3230*/5490** | 1820*/4110** | | | g/mol |
| D = M_{w}/Mₙ | 2.7*/3.7** | 2.7*/3.9** | 1.6*/2.6** | | | |

The number average molar mass Mₙ and mass average molar mass M_{w} are given by the equations as disclosed before, measured with Method A* and Method B**

TABLE 9
Product properties of the granules (inventive),
measured with Malvern Particle Size Analyzers

| | | | | | | | |
|---|---|---|---|---|---|---|---|
| Pressure | 0.2 | bar | 1.0 | bar | 3.0 | bar | bar |
| Dx (10) | 457 | μm | 441 | μm | 421 | μm | μm |
| Dx (50) | 653 | μm | 637 | μm | 612 | μm | μm |
| Dx (90) | 950 | μm | 926 | μm | 891 | μm | μm |
| D [4; 3] | 682 | μm | 663 | μm | 637 | μm | μm |
| D [3; 2] | 631 | μm | 614 | μm | 589 | μm | μm |

| | | | | |
|---|---|---|---|---|
| Width | 0.754 | 0.761 | 0.768 | |
| Uniformity | 0.234 | 0.232 | 0.234 | |
| Laser shading | 0.36 | 0.09 | 0.05 | % |
| Light shading - blue | 0.00 | 0.00 | 0.00 | % |
| Concentration | 0.0292 | 0.0068 | 0.0037 | % |
| Particle density | 1.00 | 1.00 | 1.00 | g/cm³ |
| Specific surface | 9.508 | 9.778 | 10.19 | m²/kg |

TABLE 10
Stability of the example 1-15 in liquid detergent formulation A

| | | poly- | | | | | | | | | | |
|---|---|---|---|---|---|---|---|---|---|---|---|---|
| | | acrylic | | | | | | | | | | |
| Example | | acid | 1 | 3 | 8 | 9 | 10 | 11 | 12 | 13 | 14 | 15 |
| Appearance | | 1 wt % polymer (dry) concentration | | | | | | | | | | |
| after | | in detergent formulation A | | | | | | | | | | |
| 0 | d | C | C | C | C | C | C | C | C | C | C | C |
| 7 | d | C | C | ST | C | C | C | C | C | C | C | C |
| 28 | d | C | C | ST | C | C | C | C | C | C | C | C |
| Appearance | | 3 wt % polymer (dry) concentration | | | | | | | | | | |
| after | | in detergent formulation A | | | | | | | | | | |
| 0 | d | SP | C | ST | C | ST | ST | C | C | T | C | C |
| 7 | d | SP | ST | ST | C | ST | ST | C | C | SP | C | C |
| 28 | d | SP | ST | SP | C | ST | ST | C | C | SP | C | C |

Classification:
C = clear |
ST = slightly turbid |
T = slightly turbid |
SP = separated

TABLE 11
Washing performance test of the example 9 and 14 in comparison to PA1

| hard- | | | | | | | | | | | | | | | Delta | Delta | Delta |
|---|---|---|---|---|---|---|---|---|---|---|---|---|---|---|---|---|---|
| ness | | | sum (Cotton) | | | | sum (PES) | | | | sum (total) | | | | (Cotton) | (PES) | (total) |
| [mmol/L] | waste | | % R | L | Y | DY | % R | L | Y | DY | % R | L | Y | DY | % R | % R | % R |
| | | | 436.3 | 471.3 | 429.5 | | 241.4 | 276.5 | 243.2 | | 677.6 | 747.8 | 672.7 | | | | |
| 2.5 | clayslurry | Without | 347.5 | 444.4 | 369.8 | 59.7 | 216.2 | 269.0 | 226.8 | 16.5 | 563.7 | 713.4 | 596.6 | 76.2 | | | |
| | | polymer | | | | | | | | | | | | | | | |
| | | Example 9 | 354.1 | 446.4 | 373.9 | 55.6 | 219.3 | 270.0 | 228.9 | 14.3 | 573.5 | 716.3 | 602.8 | 69.9 | 6.6 | 3.2 | 9.8 |
| | | Example 14 | 352.9 | 446.1 | 373.3 | 56.2 | 219.2 | 269.9 | 228.6 | 14.6 | 572.1 | 715.9 | 601.9 | 70.8 | 5.4 | 3.0 | 8.4 |
| | | PA1 | 353.4 | 446.2 | 373.7 | 55.8 | 215.0 | 268.7 | 226.2 | 17.0 | 568.5 | 715.0 | 599.9 | 72.8 | 5.9 | −1.1 | 4.8 |
| 2.5 | red clay/ | Without | 282.6 | 426.6 | 333.8 | 95.7 | 194.7 | 264.3 | 217.0 | 26.2 | 477.3 | 690.9 | 550.8 | 121.9 | | | |
| | SBL | polymer | | | | | | | | | | | | | | | |
| | | Example 9 | 308.0 | 436.2 | 352.9 | 76.6 | 196.4 | 264.8 | 218.1 | 25.2 | 504.4 | 701.0 | 571.0 | 101.8 | 25.4 | 1.7 | 27.1 |
| | | Example 14 | 298.3 | 432.8 | 345.9 | 83.6 | 197.6 | 265.3 | 219.0 | 24.3 | 496.0 | 698.0 | 564.9 | 107.8 | 15.8 | 3.0 | 18.7 |
| | | PA1 | 294.3 | 431.7 | 343.8 | 85.7 | 196.0 | 264.7 | 217.8 | 25.4 | 490.3 | 696.4 | 561.6 | 111.1 | 11.8 | 1.3 | 13.0 |

TABLE 12

| Water | | | Sum cotton |
|---|---|---|---|
| hardness | Soil | Tested polymer | fabrics [% R] |
| 2.5 | clayslurry | Without polymer | 347.5 |
| 2.5 | clayslurry | Example 9 | 354.1 |
| 2.5 | clayslurry | Example 14 | |
| 2.5 | clayslurry | PA1 | 353.4 |
| 2.5 | SBL Red clay fabric/SBL | Without polymer | 282.6 |
| | 2004 | | |
| 2.5 | SBL Red clay fabric/SBL | Example 9 | 308.0 |
| | 2004 | | |
| 2.5 | SBL Red clay fabric/SBL | Example 14 | |
| | 2004 | | |
| 2.5 | SBL Red clay fabric/SBL | PA1 | 294.3 |
| | 2004 | | |

TABLE 13
North America Antiredeposition performance
of examples compared to PA1

| | ΔE, Sum | ΔE, Sum | ΔE, Sum | ΔE, Sum |
|---|---|---|---|---|
| | of all | of all | of all | of all |
| | WFK 10 A | WFK 12 A | WFK 20 A | fabrics |
| Detergent Blank, | 2.00 | 6.33 | 1.89 | 10.22 |
| without polymer | | | | |
| Example 8 | 0.897 | 1.60 | 0.453 | 2.95 |
| Example 9 | 0.96 | 1.56 | 0.462 | 2.982 |
| Example 10 | 0.762 | 1.65 | 0.408 | 2.820 |
| Example 14 | 0.643 | 2.28 | 0.533 | 3.456 |
| Example 15 | 0.773 | 2.23 | 0.428 | 3.431 |
| PA1 | 1.10 | 1.87 | 0.467 | 3.437 |

TABLE 14
Accelerated aging performance of polysaccharide-graft
polymer in the presence of chelators and without

| | | Inflection time, (hr:min) given | | |
|---|---|---|---|---|
| | Wt % of | residual peroxide amount | | |
| Chelator | chelator | 0.05% | 0.10% | 0.20% |
| Without Chelator | 0.00 | 1:07 | 1:19 | 1:50 |
| DTPA-NA₅ | 0.05 | 4:25 | >6:34 | 6:20 |
| | 0.1 | 4:25 | >6:34 | >6:34 |
| | 0.25 | 4:25 | >6:34 | >6:34 |
| EDTA-Na₄* | 0.05 | 1:07 | 4:22 | 6:20 |
| | 0.1 | 1:07 | 2:37 | 6:20 |
| | 0.25 | 1:07 | 1:49 | 4:22 |
| MGDA-Na₃ | 0.025 | 1:07 | 1:25 | 1:25 |
| | 0.05 | 0:49 | 1:25 | 1:25 |
| | 0.1 | 0.49 | 1:25 | 1:25 |
| Citrate-Na₃** | 0.025 | 1:07 | 1:37 | 1:49 |
| | 0.05 | 1:25 | 1:49 | 2:20 |
| | 0.1 | 1:25 | 1:49 | 2:37 |
| EDDS-Na₃ | 0.05 | 1:16 | 2:25 | 2:44 |
| | 0.1 | 1:16 | 1:57 | 2:25 |
| | 0.25 | 1:16 | 1:16 | 1:57 |
| Glucaric acid*** | 0.05 | 2:44 | >4:00 | >4:00 |
| | 0.1 | 2:44 | 3:59 | >4:00 |
| | 0.25 | 2:44 | 3:59 | >4:00 |

*EDTA-Na₄ = ethylenediaminetetraacetic acid, tetrasodium salt
**Citrate-Na₃ = trisodium citrate
***Neutralized to pH 6.0-6.5

1-30. (canceled)

31. A water-soluble graft polymer comprising as polymer base a polysaccharide, and as grafted side chains at least one water soluble ethylenically unsaturated monomer, such unsaturated monomer comprising at least one sodium carboxylate unit, being produced by radically initiated polymerization reaction, and further employing during the polymerization at least one organic compound capable of complexing metal ions, containing in its structure at least one carboxyl-group which may be partially or fully present as acid-group or deprotonated in salt-form.

32. The graft polymer according to claim 31 wherein the polysaccharide consists of a maximum of n=30 glucose units on average.

33. The graft polymer according to claim 32 wherein the polysaccharide consists of n=2-20 glucose units on average.

34. The graft polymer according to claim 33 wherein the polysaccharide consists of n=3-10 glucose units on average.

35. The graft polymer according to claim 31 wherein the polysaccharide is selected from maltodextrin, corn syrups and/or corn syrups solids.

36. The graft polymer according to claim 31 wherein the at least one water soluble ethylenically unsaturated monomer comprises at least 50 mol. %, sodium acrylate and/or methacrylate, based on the total amount of monomers employed, and optionally other monomers polymerizable.

37. The graft polymer according to claim 31, wherein the radical polymerization is of the radical redox polymerization-type.

38. The graft polymer according to claim 31, wherein at least one organic compound is employed to reduce or prevent the discoloration before, during and/or after polymerization, such that a sufficient amount of the at least one such organic compound is still present after the polymerization and after post-polymerization.

39. The graft polymer according to claim 38, wherein as radical initiators for at least a main polymerization stage a polymerization system comprising at least one metal salt and peroxide is employed.

40. The graft polymer to according to claim 38, wherein the at least one organic compound comprises at least one functional group to form metal complexes with the at least one metal salt used as part of the polymerization-system.

41. The graft polymer according to claim 38, wherein the metal salt comprises, preferably contains only metal cations, which is most preferably employed as ferrous (II) sulfate, copper (II) sulfate, and their hydrate derivatives.

42. The graft polymer according to claim 31, wherein the at least one organic compound which is of natural origin and contains at least one carboxylate group, such as gluconic acid and their salts, preferably at least two carboxylate groups (COOR), in case of at least two carboxylate groups being more preferably separated with 1 to 3 others atoms, and even more preferably are separated with 1 to 3 others atoms which are selected from the atom types C, Si, N, P, O, S, and even more preferred have one of the following chemical Structures 1 to 4 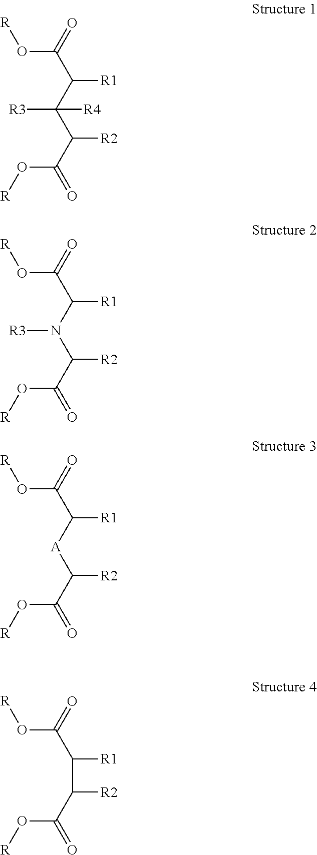
for all Structures 1 to 4 the variable being selected as follows:

| | |
|---|---|
| R | H, Li, Na, K, Ca, preferred H or Na |
| A | O or S |
| R1, R2, R3 | H and/or organic substituent containing 1 to 20 carbon-atoms and optionally |
| | further containing amino, hydroxy, carboxylic acid, sulfonic-acid, sulfonate, |
| | phosphonate, keto, aldehyde, and/or aromatic groups, H and/or the organic |
| | structure containing one or more further carboxylate groups selected from |
| | —COOR and —COOH, |
| R4 | H, OH, or organic substituent containing 1 to 20 carbon-atoms and optionally |
| | further containing amino, hydroxy, carboxylic acid, sulfonic-acid, sulfonate, |
| | phosphonate, keto, aldehyde, and/or aromatic groups, H and/or the organic |
| | structure containing one or more further carboxylate groups selected from |
| | —COOR and —COOH. |

43. The graft polymer according to claim 42 wherein the organic compound has at least three carboxylate groups (COOR).

44. The graft polymer according to claim 42, wherein the organic compound is selected from citric acid, metylglycidine di acetic acid, ethylene diamin tetra acetic acid, tetra N,N-bis(carboxylatomethyl)-glutaminic acid, di ethylene triamine penta acetic acid, tartaric acid, succinic acid, or any of their salts such as ammonium, alkali, and/or earth alkali salts.

45. The graft polymer according to claim 42, wherein the molar ratio of the total amount of the metal salt and the total amount of the at least one organic compound is between 1:1 to 1:10,000.

46. The graft polymer according to claim 42 wherein the organic compounds are chelating agents which exhibit a “Ready Biodegradability” according to the OCED Guideline (Test No. 301F).

47. The graft polymer according claim 31 in dried form or in granule or granulate form.

48. The graft polymer according to claim 31 in solution form.

49. The graft polymer according to claim 31 which fulfills at least one of the following requirements, in the order of preference b> c> d> a:
a. is readily biodegradable according to the tests as defined by OECD 301F and/or B guidelines, exhibiting a biodegradation in weight percent based on the total polymer weight of at least 50% within 28 days according to OECD 301F and/or B (based on the total solid content), and/or
b. exhibits a Gardner color less than 4.0, and/or
c. exhibits a color stability at least 6 months with an increase of maximum three Gardner color value units, and/or
d. nd the invented polymer forms a clear liquid solution in a liquid detergent formulation, without any turbidity and precipitations in the liquid detergent formulation, specifically a liquid hand dish formulation, at least 28 days by a minimum content of 0.5% of invented polymer in the liquid detergent's formulation.

50. A process to produce the graft polymer according to claim 31, wherein
a) as reactants
i) a polymer base being at least one polysaccharide, the polysaccharide consisting of a maximum of up to 30 glycose units in average, more preferably consisting of maltodextrin, corn syrups and or corn syrups solids consisting of up to 20, preferably 2 to 20, more preferably 3 to 10 glucose units on average, and
ii) at least one water soluble ethylenically unsaturated monomer, such unsaturated monomer comprising at least one sodium carboxylate unit, which may be neutralized from the carboxylic acid-form before adding to the reactor (i.e. when the polysaccharide is not present during the neutralization), in situ (in the reactor when the polysaccharide is present), wherein at least 50 mol % of the total amount of the monomer(s) employed for the polymerization reaction is sodium acrylate, and optionally at least one other polymerizable monomer, and most preferably the sodium acrylate is prepared before adding into the reaction mixture, are polymerized by radically initiated polymerization reaction,
b) further adding before or during at least the main polymerization reaction at least one organic compound containing in its structure at least two carboxyl-groups which may be partially or fully present as acid-group or deprotonated in their salt-form;
c) such polymerization reaction(s) taking place in a suitable solvent or solvent mixture, such solvent(s) being selected from polar protic or polar aprotic solvents,
d) optionally adding after the main polymerization step a further portion of the same initiator or initiator system as employed in the main polymerization stage or a different initiator or initiator system, selected from azo initiators, redox initiators, peroxides, photo-initiators and mixtures thereof, to further reduce the amount of unreacted monomers within the polymerization reaction mixture, to obtain a polymer solution comprising the graft polymer and the organic compound(s),
e) optionally subjection of the polymer solution to a further treatment selected from steam stripping, steam distillation, thermal distillation and/or vacuum distillation, to obtain a polymer solution which is purified compared to the polymer solution before this present step, and
f) optionally drying the polymer solution obtained in the previous step to obtain the graft polymer in solid form, by employing as drying means at least one spray-drier, freeze-drier, vacuum-drier, roller drum-drier, paddle drier, kneader, or any means for agglomeration such as fluidized bed-agglomeration and the like to obtain the polymer as agglomerates or granules or granulates, preferred are drying methods, which have a low temperature impact on the polymer to prevent discoloration, like spray-drier, freeze-drier, vacuum-drier, agglomeration such as fluidized bed-agglomeration, to obtain a polymer solid.

51. The process according to claim 50 wherein the polymerization takes place at a temperature of from 70 to 150, and the post-polymerisation reaction takes place at a temperature that is lower by about 5 to 10 degree compared to the temperature of the main polymerization reaction.

52. The process according to claim 50 wherein the polymerization takes place at a solids concentration based on the combined total amount of polysaccharide and monomers employed of from 20 to 60.

53. The process according to claim 50 wherein the graft polymer is produced by reacting an aqueous solution of corn syrup consisting of 3 to 8 glucose units on average with a mixture of acrylic acid and sodium acrylate with an degree of neutralization of this mixture of 90 to 96%, using a hydrogen peroxide initiator and ferrous sulfate heptahydrate redox catalyst, with the use of citric acid or their salts and preferably citric acid monohydrate or aqueous citric acid solution during the polymerization and post-polymerization to chelate metal ion(s), and using sodium persulfate in combination with hydrogen peroxide during post-polymerization to consume residual acrylate monomer, with a polymerization temperature from 90 to 100° C. and a post polymerization temperature from 85 to 95° C., with a graft polymer solids content after the complete polymerization from 20 to 60.

54. Use of at least one graft polymer according to claim 31 in a composition, that is a cleaning composition, fabric and home care product, an industrial and institutional cleaning product, cosmetic or personal care product, or agrochemical formulations.

55. The use according to claim 54 in compositions for fabric and home care, cleaning composition, or an industrial and institutional cleaning product.

56. The use according to claim 54, wherein the composition comprises at least one graft polymer at a concentration of from about 0.05% to about 10% in weight % in relation to the total weight of such composition or product.

57. The use according to claim 54, comprising
a. at least one enzyme, and/or
b. about 1% to about 70% by weight of a surfactant system, and/or
c. at least one further cleaning adjunct in effective amounts, and/or
d. exhibiting an improved washing performance in red clay dispersion in comparison to sodium polyacrylate-polymer having a Fikentscher's K-Value of 15 when substituted by the graft polymer within such composition,
preferably comprising at least one of a) to c), more preferably at least a) and b), even more preferably a), b) and c).

58. A laundry detergent composition, a cleaning composition or a fabric and home care product, preferably a laundry detergent composition, more preferably a liquid or semi-liquid or multi-component laundry detergent composition, most preferably a liquid laundry detergent composition, comprising at least one graft polymer according to claim 31, comprising the at least one graft polymer at a concentration of preferably from about 0.05% to about 10% in weight % in relation to the total weight of such composition or product, and optionally further comprising
a. at least one enzyme, and/or
b. about 1% to about 70% by weight of a surfactant system, and/or
c. at least one further cleaning adjunct in effective amounts.

59. A dish wash detergent composition, preferably a liquid dish wash detergent composition or a solid automated dish wash detergent composition, more preferably a liquid hand dish wash detergent composition, comprising at least one graft polymer claim 31, comprising the at least one graft polymer at a concentration of from about 0.05% to about 10% in weight % in relation to the total weight of such composition, and optionally further comprising
a. at least one enzyme, and/or
b. about 1% to about 70% by weight of a surfactant system, and/or
c. at least one further cleaning adjunct in effective amounts, and/or
d. at least one chelant, and/or
e. at least one anionic surfactant.
